(19) United States
(12) Patent Application Publication
(10) Pub. No.: US 2026/0234673 A1
(43) Pub. Date: Aug. 13, 2026

(54) RESPIRATION OF NANOPARTICLES BY ELECTROGENIC BACTERIA FOR PHOTO-CATALYTIC HYDROGEN EVOLUTION

(71) Applicant: University of Rochester, Rochester, NY (US)

(72) Inventors: Todd Krauss, Pittsford, NY (US); Kara L. Bren, Rochester, NY (US)

(73) Assignee: University of Rochester, Rochester, NY (US)

(21) Appl. No.: 19/119,280

(22) PCT Filed: Oct. 10, 2023
(86) PCT No.: PCT/US2023/034776

§ 371 Date: Apr. 8, 2025

Related U.S. Application Data
(60) Provisional application No. 63/414,598, filed on Oct. 10, 2022.

(51) Int. Cl. C12P 3/00; B01J 27/04; B01J 27/057; B01J 35/39; B82Y 30/00; C12N 13/00

(52) U.S. Cl. CPC C12P 3/00; B01J 27/04; B01J 27/0573; B01J 35/39; C12N 13/00; B82Y 30/00

(57) ABSTRACT

A composition that produces hydrogen includes a nanoparticle or plurality of nanoparticles; an external source of electrons such as an electrogenic bacterium or a plurality of electrogenic bacteria and a carbon source; and an aqueous medium. The nanoparticles and the aqueous medium are combined in a mixture and, upon exposure to electromagnetic radiation with a wavelength in the absorption profile of the nanoparticles, the nanoparticles generate an electron that can reduce a proton in the aqueous medium. The source of electrons is capable of reducing the nanoparticles. The nanoparticles may comprise cadmium chalcogenide or water-soluble cadmium chalcogenide quantum dots. The nanoparticles may also comprise core-shell nanoparticles, nanorods; dot-in rods, Zn-based II-VI core quantum dots, and nanoplatelets including core-crown and core-shell nanoplatelets. The electrogenic bacterium or bacteria may comprise Shewanella oneidensis, a Geobacter species or any bacterium capable of extracellular electron transfer.

### CROSS-REFERENCE TO RELATED APPLICATIONS

This application is a national stage application under 35 U.S.C. § 371 of PCT Application No. PCT/US2023/034776, filed Oct. 10, 2023, which claims the benefit of and priority to U.S. Provisional Pat. Application No. 63/414,598, filed on Oct. 10, 2022, both of which are hereby incorporated by reference in their entirety.

### STATEMENT REGARDING FEDERALLY SPONSORED RESEARCH OR DEVELOPMENT

This invention was made with government support under DE-SC0023354, DE-SC0002106, and DE-FG02-09ER16121 awarded by Department of Energy, and GM118283 awarded by the National Institutes of Health. The government has certain rights in the invention.

### 1. TECHNICAL FIELD

The present invention generally relates to production of hydrogen (i.e., H₂). More particularly, the invention relates to methods for producing hydrogen using nanoparticles and electrogenic bacteria.

### 2. BACKGROUND

One challenge facing the world is the harvesting, production, and distribution of energy to support economic prosperity with responsible environmental stewardship. For example, in 2010, energy consumption in the U.S. was at a rate of 3 TW, of which 83% originated from fossil fuels. Thus, finding reliable and robust alternatives to petroleum-based fuels would enhance U.S. energy independence and energy security.

One way to mitigate the risks of relying on liquid fossil fuels is to develop alternative sources of energy, such as biofuels or synthetic fuels. One approach is the use of biofuels (i.e., biofuel mixed with ordinary petroleum-based fuel). However, the main drawback with the use of these fuels is the cost; biofuels can cost about 10 times as much as petroleum-based fuels. Currently, the synthesis of these new biofuels often involves the hydrogenation of animal or vegetable fats as a step in the production of liquid fuel. Long-term, however, a drawback to the widespread use of such biofuels is the cost to produce the hydrogen involved in the hydrogenation process. Hydrogen is typically produced through high-temperature steam reforming of hydrocarbons such as methane or liquid fuels. This process is energy intensive and also relies on natural gas, which is a fossil fuel, and thus the production of hydrogenated fuels will suffer to a great extent from the same risks as petroleum based fuels. Furthermore, a recent study concluded that the complete life-cycle process of biofuels synthesis actually increases greenhouse gas emissions.

The successful utilization of the clean energy carrier hydrogen (H₂) in the synthesis of biofuels, synthetic fuels (i.e., from syngas), or even as a fuel itself, requires methods for H₂ production using primary energy sources not based on fossil fuels. Of possible primary energy sources, solar offers the greatest long-term impact because of its abundance and availability, but many challenges need to be met for its utilization. For the direct conversion of sunlight to stored chemical energy in H₂, efficient photochemical reduction of protons in water is needed.

Molecular hydrogen can be produced from protons (H⁺) in the reductive half-reaction of artificial photosynthesis (AP) systems. One of the strategies for light-driven proton reduction features a multicomponent solution with a light-absorbing molecule (chromophore) that transfers electrons to a catalyst that reduces protons. However, these solution systems often use nonaqueous solvents, and always have short lifetimes from decomposition of the chromophore over a period of hours. This difficulty has led to more complicated architectures that separate the sites of light absorption and proton reduction. Heterostructures between nanocrystals (NCs) and traditional precious metal nanoparticle H₂ production catalysts, and between NCs and iron-hydrogenases, have produced proton reduction in solution.

Research in artificial photosynthesis has been active since the 1970's. The energy storing reaction that is of greatest importance in artificial photosynthesis is the decomposition of water into its constituent elements, H₂ and O₂, with the former as the fuel. As a redox reaction, water splitting can be divided into its two half-cell components for separate investigation and development. Despite great efforts over the past decade, neither half-reaction has been carried out photochemically in a system composed of earth-abundant elements with both an activity and robustness of the type needed for further development.

One approach for solar energy conversion relies on systems designed and built entirely on the molecular level, either to use light to achieve chemical potential in the form of charge separation, or as systems for solar fuel generation. Entirely molecular systems can present synthetic challenges, and molecular chromophores are prone to photodegradation. Reduced photosensitizers are unstable and rapidly photobleach, thus limiting the amount of H₂ potentially produced. Organic photosensitizers can store and therefore deliver only one electron at a time, while two are required for H₂ production. Thus, the turnover frequency for the overall catalytic processes is relatively slow, being dependent on diffusion of two photoexcited sensitizers to the catalyst. This second problem can be somewhat mitigated with a higher photon flux; however, a higher excitation rate exacerbates the photobleaching problem. Homogeneous systems for light-driven reduction of protons to H₂ typically suffer from short lifetimes because of decomposition of the light-absorbing molecule and/or catalyst, if present.

Developing systems that interface nanomaterials and microorganisms is an attractive approach to artificial photosynthesis that may take advantage of the extraordinary properties of both components.

### 3. SUMMARY

A first aspect of the disclosed invention is directed to a composition for producing hydrogen, comprising: a nanoparticle or plurality of nanoparticles; an external source of electrons, wherein the external source of electrons is an electrogenic bacterium or a plurality of electrogenic bacteria and a carbon source; and an aqueous medium; wherein the nanoparticle or the plurality of nanoparticles and the aqueous medium are combined in a mixture; upon exposure to electromagnetic radiation with a wavelength in the absorption profile of the nanoparticle or the plurality of nanoparticles, the nanoparticle or the plurality of nanoparticles is capable of generating an electron that can reduce a proton in the aqueous medium; and the source of electrons is capable of reducing the nanoparticle or the plurality of nanoparticles.

The nanoparticles or plurality of nanoparticles may be selected from the group consisting of: (1) CdSe, CdS, CdTe, PbS, PbTe, PbSe, GaAs, InP, InAs, Si, and Ge nanoparticles; (2) core-shell nanoparticles, including CdSe/CdS, CdSe/CdTe, CdTe/CdSe, and PbSe/PbS core-shell nanoparticles; (3) CdSe, CdS, CdTe, PbS, PbTe, PbSe, GaAs, InP, InAs, Si, and/or Ge nanorods; (4) dot-in rods, including CdSe/CdS, CdSe/CdTe, CdTe/CdS, and CdTe/CdSe dot-in rods; (5) Zn-based II-VI core QDs, (6) nanoplatelets including core-crown and core-shell nanoplatelets comprising CdSe, CdS, CdTe, PbS, PbTe, PbSe, GaAs, InP, InAs, Si, Ge, SnS, SnSe, SnTe, Ag2S, Ag2Se and/or Ag2Te; (7) SnS, SnSe, SnTe, Ag2S, Ag2Se and Ag2Te; and combinations thereof. Preferably, the nanoparticle or the plurality of nanoparticles comprise cadmium chalcogenide (CdSe or CdS). Preferably, the nanoparticle or the plurality of nanoparticles comprise water-soluble cadmium chalcogenide (CdSe or CdS) quantum dots. Quantum dots with varied sizes (between 2-10 nm) can be used. Changing the quantum dot size changes the wavelength of light absorbed and the energies of the conduction and valence bands. A more reducing conduction band may yield more active catalysis, and a more oxidizing valence band may enhance EET from bacteria.

The nanoparticles may also be ternary or quaternary compounds. Cu-based ternary or quaternary quantum dots (QDs), or semiconductor nanocrystals (NCs) have unique optical properties regarding their emission mechanism, high photoluminescent quantum yields (PLQYs), size-dependent bandgap, composition-dependent bandgap, broad emission range, large Stokes' shift, and long photoluminescent (PL) lifetimes. Various types of Cu-based ternary or quaternary QDs (including anisotropic NCs, polytypic NCs, and spherical, nanorod and tetrapod core/shell heterostructures) may be used. For example, ternary Cu—In—Se (CISe) and Cu—In—S(CIS) compounds may be used, such as CuInS₂ and CuInSe₂, or Cu—Sn—Se (CTSe) and Cu—Sn—S(CTS) compounds may be used. Variations of these structures and their synthesis are detailed in Bai, et al. Optical Properties, Synthesis, and Potential Applications of Cu-Based Ternary or Quaternary Anisotropic Quantum Dots, Polytypic Nanocrystals, and Core/Shell Heterostructures Nanomaterials (Basel). 2019 January; 9 (1): 85, Published online 2019 Jan. 10. doi: 10.3390/nano9010085. Any combination of II-VI, III-V and I-VII elements that have the correct stoichiometry could be used.

Additionally, metal halide perovskite NCs have attractive optical and electronic properties along with low cost and solution processability. Halide perovskites have a general formula of ABX₃, where A and B are monovalent and divalent cations, respectively, and X is a monovalent halide (Cl, Br, I) anion. Lead halide perovskites, classified into either organic-inorganic (hybrid) or all-inorganic, depending on whether the A cation is an organic molecule. Most commonly used is methylammonium (MA, CH3NH3+) for a hybrid compound, or an inorganic cation (commonly Cs+), may be used for the inorganic compound. Examples are MAPbX₃ nanoparticles and caesium lead halide perovskite NCs (CsPbX₃) where X═Cl, Br or I. Variations of these structures and their synthesis are detailed in Huang, H., Polavarapu, L., Sichert, J. et al. Colloidal lead halide perovskite nanocrystals: synthesis, optical properties and applications. NPG Asia Mater 8, e328 (2016). https://doi.org/10.1038/am.2016.16.

Preferably, the electrogenic bacterium or plurality of electrogenic bacteria may comprise Shewanella oneidensis, a Geobacter species or a genetically-modified bacterium. Any bacterium capable of extracellular electron transfer may also be used. Shewanella oneidensis MR-1 in which genes for both hydrogenase enzymes are knocked out (ΔhydA/ΔhyaB) can be used, with the advantage that without their own functional hydrogenases, these strains will not consume hydrogen produced by the system. Shewanella oneidensis in which genes expressing proteins that perform extracellular electron transfer are overexpressed may also be used, with the advantage that these are expected to yield a greater flux of electrons to the quantum dots. The genes to be overexpressed include CymA, MtrA, MtrB, OmcA, MtrC.

In one aspect in the invention, the nanoparticle or the plurality of nanoparticles comprise water-soluble cadmium chalcogenide (CdSe or CdS) quantum dots, and the electrogenic bacterium or plurality of electrogenic bacteria comprise Shewanella oneidensis, a Geobacter species or a genetically-modified bacterium.

In another aspect of the invention, the electromagnetic radiation to which the composition is exposed has a wavelength of between approximately 400 and 1100 nanometers (nm). More preferably, the electromagnetic radiation may have a wavelength of approximately 530 nm.

In another aspect of the invention, the pH of the mixture is approximately 7.

In one embodiment of the invention, the nanoparticle or the plurality of nanoparticles comprise water-soluble cadmium chalcogenide (CdSe or CdS) quantum dots, the electrogenic bacterium or plurality of electrogenic bacteria comprise Shewanella oneidensis, a Geobacter species or a genetically-modified bacterium, the electromagnetic radiation has a wavelength of approximately 530 nm and the pH of the mixture is approximately 7.

In one aspect of the invention, the aqueous medium may be wastewater. In another aspect of the invention, the aqueous medium may be water.

In one aspect of the invention, the carbon source comprises lactate.

In any of the embodiments of the above aspect of the invention, the electrogenic bacterium or plurality of electrogenic bacteria may preferably comprise Shewanella oneidensis and have at least one mutation selected from the group consisting of (1) deletion of the genes HydA and/or HyaB, encoding hydrogenases; (2) deletion of any of the hydrogenase-related genes, or any combination of HydA, HydB, HydE, HydF, and/or HydG; (3) deletion or overexpression of any of the genes associated with extracellular electron transfer, including OmcA, MtrA, MtrB, MtrC, and/or CymA; (4) deletion or overexpression of any of the genes associated with extracellular electron transfer mediated by flavins and/or flavin biosynthesis, including RibF, RibDBAE, and/or OprF; (5) deletion or overexpression of any of the genes associated with biofilm formation including RibF and/or YdeH; and (6) display of surface peptides containing amino acids that may bind to nanoparticles, including histidine and cysteine.

More preferably, the mutation is deletion of the genes HydA and/or HyaB, encoding hydrogenases, or the mutation is deletion or overexpression of any of the genes associated with extracellular electron transfer, including OmcA, MtrA, MtrB, MtrC, and/or CymA.

A second aspect of the invention is directed to a method for producing hydrogen comprising: (a) providing a source of electrons wherein the source of electrons is an electrogenic bacterium or a plurality of electrogenic bacteria and a carbon source; (b) contacting a nanoparticle or a plurality of nanoparticles in an aqueous medium to form a mixture in the presence of the source of electrons; and (c) exposing the mixture from (b) to electromagnetic radiation having at least a wavelength in the absorption profile of the nanoparticle or the plurality of nanoparticles, wherein: upon exposure to the electromagnetic radiation, the nanoparticle or the plurality of nanoparticles is capable of generating an electron that can reduce a proton in the aqueous medium, and the source of electrons is capable of reducing the nanoparticle or the plurality of nanoparticles, such that hydrogen is produced.

In one embodiment of the method, the nanoparticle or the plurality of nanoparticles may be selected from the group consisting of: (1) CdSe, CdS, CdTe, PbS, PbTe, PbSe, GaAs, InP, InAs, Si, and Ge nanoparticles; (2) core-shell nanoparticles, including CdSe/CdS, CdSe/CdTe, CdTe/CdSe, and PbSe/PbS core-shell nanoparticles; (3) CdSe, CdS, CdTe, PbS, PbTe, PbSe, GaAs, InP, InAs, Si, and/or Ge nanorods; (4) dot-in rods, including CdSe/CdS, CdSe/CdTe, CdTe/CdS, and CdTe/CdSe dot-in rods; (5) Zn-based II-VI core QDs, (6) nanoplatelets including core-crown and core-shell nanoplatelets comprising CdSe, CdS, CdTe, PbS, PbTe, PbSe, GaAs, InP, InAs, Si, Ge, SnS, SnSe, SnTe, Ag2S, Ag2Se and/or Ag2Te; (7) SnS, SnSe, SnTe, Ag2S, Ag2Se and Ag2Te; and combinations thereof. Preferably, the nanoparticle or the plurality of nanoparticles comprise cadmium chalcogenide (CdSe or CdS). Preferably, the nanoparticle or the plurality of nanoparticles comprise water-soluble cadmium chalcogenide (CdSe or CdS) quantum dots.

In one aspect of the method, the electrogenic bacterium or plurality of electrogenic bacteria comprise Shewanella oneidensis, a Geobacter species or a genetically-modified bacterium. In another aspect of the method, the nanoparticle or the plurality of nanoparticles comprise water-soluble cadmium chalcogenide (CdSe or CdS) quantum dots, and the electrogenic bacterium or plurality of electrogenic bacteria comprise Shewanella oneidensis, a Geobacter species or a genetically-modified bacterium.

In one aspect of the method, the electromagnetic radiation has a wavelength of between approximately 400 and 1100 nanometers (nm). Preferably, the electromagnetic radiation has a wavelength of approximately 530 nm.

The method may occur in one aspect where the pH of the mixture is approximately 7.

In one aspect of the method, the nanoparticle or the plurality of nanoparticles comprise water-soluble cadmium chalcogenide (CdSe or CdS) quantum dots, the electrogenic bacterium or plurality of electrogenic bacteria comprise Shewanella oneidensis, a Geobacter species or a genetically-modified bacterium, the electromagnetic radiation has a wavelength of approximately 530 nm and the pH of the mixture is approximately 7.

In one aspect of the method, the aqueous solution comprises wastewater. In another aspect of the method, the aqueous solution comprises water.

In one embodiment of the method, carbon source comprises lactate.

In any of the above embodiments of the method, the electrogenic bacterium or plurality of electrogenic bacteria may preferably comprise Shewanella oneidensis and have at least one mutation selected from the group consisting of (1) deletion of the genes HydA and/or HyaB, encoding hydrogenases; (2) deletion of any of the hydrogenase-related genes, or any combination of HydA, HydB, HydE, HydF, and/or HydG; (3) deletion or overexpression of any of the genes associated with extracellular electron transfer, including OmcA, MtrA, MtrB, MtrC, and/or CymA; (4) deletion or overexpression of any of the genes associated with extracellular electron transfer mediated by flavins and/or flavin biosynthesis, including RibF, RibDBAE, and/or OprF; (5) deletion or overexpression of any of the genes associated with biofilm formation including RibF and/or YdeH; and (6) display of surface peptides containing amino acids that may bind to nanoparticles, including histidine and cysteine.

More preferably, the mutation is deletion of the genes HydA and/or HyaB, encoding hydrogenases or deletion or overexpression of any of the genes associated with extracellular electron transfer, including OmcA, MtrA, MtrB, MtrC, and/or CymA.

A third aspect of the invention is directed to a composition for producing hydrogen, comprising: a nanoparticle or plurality of nanoparticles; an external source of electrons, wherein the external source of electrons is an electrogenic bacterium or a plurality of electrogenic bacteria and a carbon source; a surface; and an aqueous medium; wherein the nanoparticle or the plurality of nanoparticles adhere to the surface and the surface is in contact with the aqueous medium; upon exposure to electromagnetic radiation with a wavelength in the absorption profile of the nanoparticle or the plurality of nanoparticles, the nanoparticle or the plurality of nanoparticles is capable of generating an electron that can reduce a proton in the aqueous medium; and the source of electrons is capable of reducing the nanoparticle or the plurality of nanoparticles.

In one aspect of this embodiment, wherein the surface is an electrode or is comprised of quantum dots and at least one electrogenic bacterium adheres to the surface. Preferably, the electrogenic bacteria may form a biofilm or may reside in a biofilm.

In one aspect of this embodiment, the nanoparticle or the plurality of nanoparticles may be selected from the group consisting of: (1) CdSe, CdS, CdTe, PbS, PbTe, PbSe, GaAs, InP, InAs, Si, and Ge nanoparticles; (2) core-shell nanoparticles, including CdSe/CdS, CdSe/CdTe, CdTe/CdSe, and PbSe/PbS core-shell nanoparticles; (3) CdSe, CdS, CdTe, PbS, PbTe, PbSe, GaAs, InP, InAs, Si, and/or Ge nanorods; (4) dot-in rods, including CdSe/CdS, CdSe/CdTe, CdTe/CdS, and CdTe/CdSe dot-in rods; (5) Zn-based II-VI core QDs, (6) nano-platelets including core-crown and core-shell nanoplatelets comprising CdSe, CdS, CdTe, PbS, PbTe, PbSe, GaAs, InP, InAs, Si, Ge, SnS, SnSe, SnTe, Ag2S, Ag2Se and/or Ag2Te; (7) SnS, SnSe, SnTe, Ag2S, Ag2Se and Ag2Te; and combinations thereof. Preferably, the nanoparticle or the plurality of nanoparticles comprise cadmium chalcogenide (CdSe or CdS). Preferably, the nanoparticle or the plurality of nanoparticles comprise water-soluble cadmium chalcogenide (CdSe or CdS) quantum dots.

In another aspect of this embodiment, the electrogenic bacterium or plurality of electrogenic bacteria comprise Shewanella oneidensis, a Geobacter species or a genetically-modified bacterium.

In one aspect of this embodiment, the nanoparticle or the plurality of nanoparticles comprise water-soluble cadmium chalcogenide (CdSe or CdS) quantum dots, and the electrogenic bacterium or plurality of electrogenic bacteria comprise Shewanella oneidensis, a Geobacter species or a genetically-modified bacterium.

In one aspect of this embodiment, the electromagnetic radiation has a wavelength of between approximately 400 and 1100 nanometers (nm). Preferably, the electromagnetic radiation has a wavelength of approximately 530 nm.

In one aspect of this embodiment, the pH of the medium may be approximately 7.

In one embodiment of the invention, the nanoparticle or the plurality of nanoparticles comprise water-soluble cadmium chalcogenide (CdSe or CdS) quantum dots, the electrogenic bacterium or plurality of electrogenic bacteria comprise Shewanella oneidensis, a Geobacter species or a genetically-modified bacterium, the electromagnetic radiation has a wavelength of approximately 530 nm and the pH of the mixture is approximately 7.

In the third aspect of the invention, the aqueous medium comprises wastewater. In another embodiment, the aqueous medium comprises water.

In the third aspect of the invention, the carbon source comprises lactate.

In any of the above embodiments of the third aspect of the invention, the electrogenic bacterium or plurality of electrogenic bacteria may preferably comprise Shewanella oneidensis and have at least one mutation selected from the group consisting of (1) deletion of the genes HydA and/or HyaB, encoding hydrogenases; (2) deletion of any of the hydrogenase-related genes, or any combination of HydA, HydB, HydE, HydF, and/or HydG; (3) deletion or overexpression of any of the genes associated with extracellular electron transfer, including OmcA, MtrA, MtrB, MtrC, and/or CymA; (4) deletion or overexpression of any of the genes associated with extracellular electron transfer mediated by flavins and/or flavin biosynthesis, including RibF, RibDBAE, and/or OprF; (5) deletion or overexpression of any of the genes associated with biofilm formation including RibF and/or YdeH; and (6) display of surface peptides containing amino acids that may bind to nanoparticles, including histidine and cysteine.

More preferably, the mutation is deletion of the genes HydA and/or HyaB, encoding hydrogenases or deletion or overexpression of any of the genes associated with extracellular electron transfer, including OmcA, MtrA, MtrB, MtrC, and/or CymA.

### 4. BRIEF DESCRIPTION OF THE DRAWINGS

Embodiments are described herein with reference to the accompanying drawings, in which similar reference characters denote similar elements throughout the several views. It is to be understood that in some instances, various aspects of the embodiments may be shown exaggerated, enlarged, exploded, or incomplete to facilitate an understanding of the invention.

FIG. 1A schematic outlining the light-driven respiration of nanocrystals (NCs) by bacteria to produce hydrogen.

FIG. 2 Hydrogen evolution from CdSe-MPA (1 μM) with WT (initial OD₆₀₀=0.05, as labelled) and ΔhyaA MR-1 (initial OD₆₀₀=0.05, as labelled) was monitored under constant irradiation (530 nm) at 25° C., pH 7. Hydrogen evolution in the absence of CdSe-MPA was also recorded. A) Total H₂ evolved was measured after one week (168 hours). Error bars represent the standard deviation from ten replicate experiments. B) In addition, H₂ was monitored over time (0 to 168 hours). Error bars represent the range of values observed at each time point for triplicate experiments.

FIGS. 3A-B. A) Total H₂ evolution from WT (initial OD 0.05) and ΔhyaA MR-1 (initial OD₆₀₀=0.05) with CdSe-MPA (1 μM) as monitored in the light (530 nm) and dark after one week at 25° C., pH 7. Error bars represent the range observed in triplicate experiments. B) After a week-long catalysis with WT (initial OD₆₀₀=0.05) and ΔhyaA MR-1 (initial OD₆₀₀=0.05) with and without CdSe-MPA (1 μM), the mass accumulated was measured (method described in FIG. 10.)

FIG. 4 Hydrogen produced by the MR-1: CdSe-MPA system in MM, with fresh MM added to A) CdSe-MPA (1 μM) with WT (initial OD₆₀₀=0.05, as labelled) and B) ΔhyaA MR-1 (initial OD₆₀₀=0.05, as labelled) after 168 hours

FIG. 5. Absorbance spectrum of CdSe-MPA QDs in water. The absorbance spectrum of CdSe-MPA in H₂O after ligand exchange is shown.

FIG. 6. Hydrogen evolution from CdSe-MPA in MM. Total hydrogen evolved from CdSe-MPA (1 μM) in MM, irradiated with green LEDs (530 nm) at 25° C.

FIG. 7. Schematic of estimated redox potentials in the hydrogen evolution system. Shewanella oneidensis MR-1 (MR-1) has been posited to have multiple electron transfer pathways, mediated by direct electron transfer from outer membrane cytochromes (MtrC, OmcA) or excreted flavins as electron transfer agents. Shown here is the observed onset potential of catalysis at a carbon paste electrode (+0.2 V) which falls within other estimates made for electron transfer from MR-1 (between approximately −0.1 V and +0.3 V) representing the potential at which EET is expected to occur.

FIG. 8. The rate of hydrogen evolution from WT and CdSe-MPA. The most linear portion of the kinetics of hydrogen evolution, between 12 and 72 hours, were used to gauge the rates of hydrogen evolution from WT MR-1 with and without CdSe-MPA.

FIG. 9. The rate of hydrogen evolution from ΔhyaA and CdSe-MPA. The most linear portion of the kinetics of hydrogen evolution, between 12 and 72 hours, were used to gauge the rates of hydrogen evolution from ΔhyaA with and without CdSe-MPA.

FIG. 10. Hydrogen evolution in the light and dark. Hydrogen evolution from WT (initial OD₆₀₀ of 0.05, as labelled) and ΔhyaA (initial OD₆₀₀ of 0.05, as labelled) with CdSe-MPA (1 μM) was monitored in the light (green LEDs (530 nm)) and dark over the course of one week at 25° C.

FIG. 11. Full figure of wet masses post-photocatalysis in the light and dark. A breakdown of mass accumulated after all conditions in the light and dark (FIG. 3, FIG. 5) demonstrates that the same trends are observed in the dark as the light.

FIG. 12. Hydrogen evolution after replenishing MM with and without CdSe-MPA. After 168 hours, fresh MM was added to A) CdSe-MPA (1 μM) with WT (initial) (orange) and WT (initial OD₆₀₀ of 0.05) in the absence of CdSe-MPA (gray). B) Fresh MM was added to CdSe-MPA (1 μM) and DhyaA MR-1 (initial OD₆₀₀ of 0.05) (purple) and in the absence of CdSe-MPA (gray).

FIG. 13. Hydrogen evolution from precipitated CdSe-MPA. ΔhyaA (initial OD₆₀₀ of 0.05 was introduced to precipitated CdSe-MPA (1 μM) in solution (MM) after 72 hours and monitored in the light (green LEDs (530 nm)) at 25° C. The total hydrogen evolved from the precipitated QDs (red, solid line) is less than when using colloidal CdSe-MPA (1 μM) and ΔhyaA (initial OD₆₀₀ of 0.05) (orange, dotted line) under the same conditions.

FIGS. 14A-C. Assemblies for light-driven bio-assisted H₂ production. (A) Combination of QDs and bacteria in solution. EET may be direct or via a mediator (M/M-). (B) Separation of QDs and bacteria with a porous membrane. (C) Use of QDs and bacteria in a photo-microbial electrochemical cell.

FIG. 15. Fluorescence microscopy images demonstrating the presence of CdSe-MPA and MR-1 in MR-1: CdSe-MPA aggregates.

FIG. 16. Effect of heat killing of ΔhyaA on system hydrogen evolution activity. (A) Hydrogen evolution from ΔhyaA (initial OD₆₀₀ of 0.05, as labelled) with CdSe-MPA (1.0 μM) was monitored in the light (green LEDs (530 nm)) over the course of one week at 25° C. To demonstrate the role of bacterial metabolism in driving the reaction, heat was applied at 100° C. for 10 minutes at time points of 0 hours and 72 hours (as labelled). When heat was applied to kill the bacteria, hydrogen evolution plateaued. (B) Hydrogen evolution from CdSe-MPA (1.0 mM) in the presence of ascorbic acid (300 mM) as an electron donor after 168 hours of irradiation. “Heat killed” CdSe-MPA samples were subjected to the same heating as samples containing ΔhyaA at the start of experiment (t=0). “Heat killed” CdSe-MPA maintains high activity in the presence of a functioning external electron donor, supporting the hypothesis that the loss of activity seen for heat-killed ΔhyaA: CdSe-MPA originates from killing the ΔhyaA.

FIG. 17. Hydrogen evolution at different CdSe-MPA Concentrations. Total hydrogen evolution after 168 hr of irradiation (530 nm) with ΔhyaA (Initial OD₆₀₀ of 0.05) with 0.0, 0.5, 1.0, 1.5, and 2.0 μM CdSe-MPA at pH 7.0, 25° C. Error bars represent the standard deviation of at least two replicates. At concentrations higher than 1.0 μM CdSe-MPA, ΔhyaA cultures are sensitive to increased QD concentration and no hydrogen is observed. These inactive samples also show no aggregate formation.

FIG. 18. Time course of lactate consumption and H₂ production. Lactate concentration measured over time during 212 hours of irradiation (with ΔhyaA initial OD₆₀₀ 0.05) CdSe-MPA (1.0 μM) in MM at 25° C., pH 7). Error bars represent standard deviation of four replicates. The plateau in H₂ production aligns with lactate depletion. These data support the model that lactate is serving as the primary electron source enabling H₂ production by CdSe-MPA.

FIG. 19. OD and colony forming unit analysis over time. Optical density and colony forming unit (CFU) data were collected during 530-nm irradiation by sacrificing vials at specific timepoints. Vials contained ΔhyaA (initial OD₆₀₀ 0.05), 1.0 mM CdSe-MPA and MM. OD measurements were obtained by gently swirling the vial to break up and suspend the aggregate. CFU were quantified using the plating procedure outlined above in the SI Text and measured only the aggregate.

FIG. 20. Electron microscopy images of the MR-1/CdSe-MPA aggregate. After one week of a photochemical H₂ production experiment using WT (initial OD 0.05) and CdSe-MPA (1.0 mM), a sample of the aggregate formed was fixed with 2.5% glutaraldehyde/0.1 M sodium cacodylate solution. Images were collected on a Hitachi 7650 Transmission Electron Microscope with EDS (IF Instruments).

FIG. 21 Effect of gene knockouts on hydrogen production from MR-1/CdSe system. The total hydrogen produced after 168 hours of irradiation with 530-nm LEDs is shown for 1 mM CdSe-MPA in minimal medium with the designated strains of Shewanella oneidensis.

### 5. DETAILED DESCRIPTION

Methods and materials are provided for photoinduced charge transfer and production of hydrogen (i.e., H₂). Provided is an advance in the realm of light-to-chemical energy conversion and artificial photosynthesis. Provided is a highly active and robust aqueous system for the photogeneration of hydrogen from water using nanoparticles (NPs) and a source of electrons (e.g., a biological system).

Nanomaterials have electronic and catalytic properties depending on their composition, structure, size, and shape, along with exceptional photostability. Microorganisms carry out a range of valuable reactions while exhibiting growth and repair. Nanomaterials have been utilized to provide electrons to microorganisms in photo (electro) chemical processes to fuel metabolic pathways for desired products. Using nanomaterials to sustain the metabolic pathways of microorganisms is one route for solar-to-fuel reactivity. This strategy is limited to using the natural metabolic products of the microorganism or requires installing new activity via genetic engineering, which can place strain on the organism. In addition, directing electrons to the microorganisms fails to fully capitalize on the excellent catalytic properties of nanomaterials.

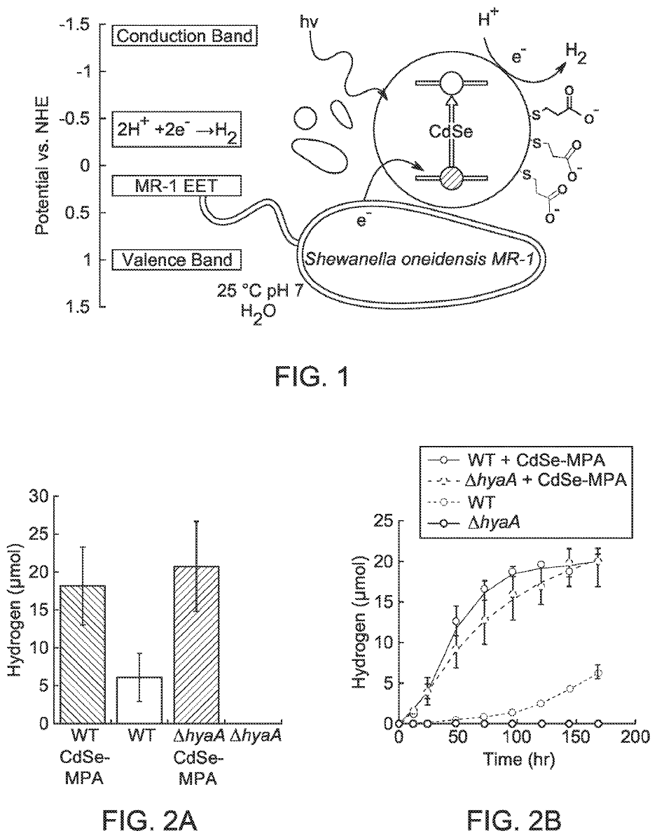

Rather than catalysis regulated by electron transfer to the microorganism, photocatalytic activity of semiconductor nanocrystals (NCs) may be sustained by extracellular electron transfer (EET) from electrogenic bacteria (FIG. 1). In this approach, the catalytic reaction is not governed by microbial metabolism, but rather by the nanomaterial. While EET from electrogenic bacteria to inorganic bulk materials and/or nanomaterial mediators has been employed within electrochemical and photoelectrochemical systems to yield current as a product, no reported systems have fueled light-driven catalysis directly with microbial EET to NCs.

In one embodiment of the invention, nanoparticles are mixed in an aqueous medium. Electrogenic bacteria provide an external source of electrons. Upon exposure to electromagnetic radiation with a wavelength in the absorption profile of the nanoparticles, the nanoparticles are capable of generating an electron that can reduce a proton in the aqueous medium. The source of electrons is capable of reducing the nanoparticles. An electron transfer from the bacteria replenishes the nanoparticles for H₂ evolution. This process occurs in the absence of an added complex metal catalyst, defined as a metal complex: (1) that can accept an electron from a photoexcited nanoparticle; (2) does not occur naturally in the aqueous medium or source of electrons. An example in U.S. Pat. No. 10,047,443 shows hydrogen production from systems containing CdSe-DHLA, Ni (NO₃)₂ and ascorbic acid (1.0 M) in H₂O (5.0 mL) at pH 4.5 upon irradiation with 520 nm light. In a typical experiment, production of hydrogen occurred upon photolysis of a solution formed from nickel (II) nitrate and NCs in water. In this example, the nickel (II) nitrate is the metal complex catalyst.

Another embodiment of the invention is a method for producing hydrogen comprising: (a) providing a source of electrons such electrogenic bacteria and a carbon source; (b) contacting nanoparticles in an aqueous medium to form a mixture in the presence of the source of electrons; and (c) exposing the mixture from (b) to electromagnetic radiation having at least a wavelength in the absorption profile of the nanoparticles. Upon exposure to the electromagnetic radiation, the nanoparticles are capable of generating an electron that can reduce a proton in the aqueous medium, and the source of electrons is capable of reducing the nanoparticles, such that hydrogen is produced.

### EXAMPLE

Cadmium chalcogenide (CdSe, CdS) NCs are exceptional photocatalysts for transformation of small-molecule organic substrates, reduction of carbon dioxide, and the reduction of protons to produce hydrogen (H₂). An energy-dense and clean fuel, H₂ is the end-product of the reductive water splitting half-reaction and a highly sought-after artificial photosynthetic product. Despite the outstanding activity of CdSe photocatalysts for H₂ evolution, a longstanding limitation of QD catalysis is the oxidative side of the reaction. Direct water oxidation is thermodynamically challenging and inefficient, and consequently, easily oxidized electron donors are typically supplemented in high concentrations to drive photocatalysis, a major limitation in their use.

It is disclosed that electrogenic bacteria may respire nanocrystals to support solar hydrogen production. An embodiment is Shewanella oneidensis MR-1 providing electrons to a CdSe nanocrystalline photocatalyst, enabling visible light-driven hydrogen production from water. Shewanella oneidensis MR-1 respires nano-crystalline CdSe quantum dots (QDs), via EET. FIG. 1 outlines light-driven respiration of NCs by bacteria to produce hydrogen. Electron transfer from MR-1 can fill a hole in the valence band of CdSe, replenishing electrons for catalytic H₂ evolution. The reaction takes place without the addition of a complex metal catalyst.

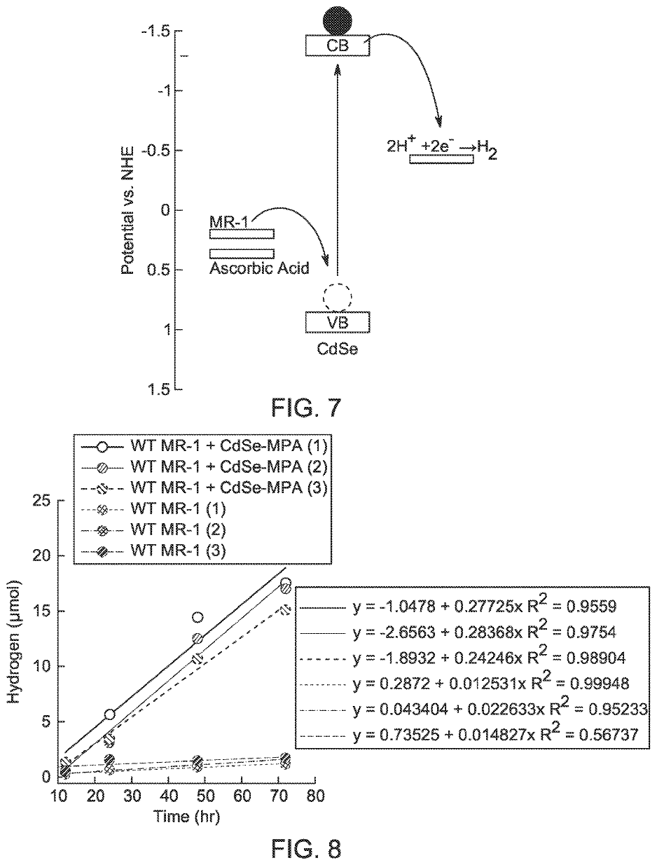

FIG. 7 is a schematic of estimated redox potentials in the hydrogen evolution system. Shewanella oneidensis MR-1 (MR-1) has been posited to have multiple electron transfer pathways, mediated by direct electron transfer from outer membrane cytochromes (MtrC, OmcA) or excreted flavins as electron transfer agents. FIG. 7 shows the observed onset potential of catalysis at a carbon paste electrode (+0.2 V) which falls within other estimates made for electron transfer from MR-1 (between approximately −0.1 V and +0.3 V), representing the potential at which EET is expected to occur. A combination of electron transfer pathways may be active. The oxidation of ascorbic acid is estimated at a similar potential to EET from MR-1, approximately +0.35 V. Both are capable of filling a hole in the valence band of CdSe, estimated at a potential of approximately +1.0 V. The thermodynamic potential for proton reduction (−0.41 V at pH 7.0) is more positive than the conduction band of CdSe, estimated to be at approximately −1.4 V, and CdSe-MPA has previously been demonstrated as a photocatalyst for the hydrogen evolution reaction.

Unlike microbial electrolysis cells, the above system requires no external potential. Illuminating this system at 530 nm yields continuous H₂ generation for >164 hours, which can be lengthened further by replenishing bacterial nutrients. EET from MR-1 is a natural anaerobic respiratory process. Replenishing the growth medium allows catalysis to continue over a second week. There seemed many possible barriers to MR-1 EET directly to colloidal QDs: surface area needed for bacterial adhesion, the oxidative damage from photocatalytic processes, biocompatibility of the NCs, and efficiency of electron transfer. The strategy reported herein demonstrates that these challenges are surmountable in a remarkably simple system and suggests a new solution to the oxidative road-block in QD photocatalysis. This strategy may be paired with a host of different NC photocatalytic reactions to yield sustainable systems for NC-mediated photocatalysis.

### Creation of the System

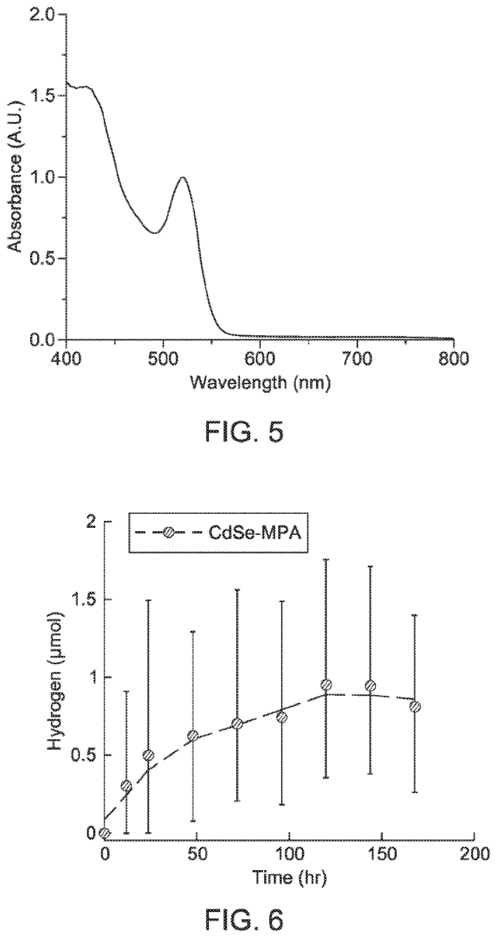

CdSe QDs were synthesized with a diameter of 2.6 nm as defined by the wavelength of the peak in their lowest energy excitonic absorption feature (525 nm, FIG. 5, where the CdSe show a well defined first exciton absorbance consistent with those shown previously). These QDs were made water soluble by exchanging their native ligands for 3-mercaptopropionic acid (MPA), yielding CdSe-MPA.

CdSe-MPA QDs can directly catalyze the proton reduction reaction in water containing ascorbic acid as a sacrificial electron donor. In modified minimal medium (MM) used for MR-1 culture, CdSe-MPA produced negligible H₂ under constant irradiation (<2 μmol), indicating there are not suitable electron donors present in the medium to support photocatalytic H₂ production. FIG. 6 shows total hydrogen evolved from CdSe-MPA (1 μM) in MM, irradiated with green LEDs (530 nm) at 25° C. MM does not contain a high excess of electron donors that CdSe-MPA can utilize directly, as evidenced from the low hydrogen evolved over the course of a week (<2 μmol).

Previous electrochemical studies of MR-1 indicate that that EET from MR-1 to electrodes occurs at a more negative reduction potential than the valence band potential of CdSe-MPA (FIG. 1, FIG. 7), suggesting that EET from MR-1 to photoexcited CdSe-MPA is thermodynamically favorable. H₂ production by CdSe-MPA QDs was measured in wild-type MR-1 (WT) cultures. Cultures were grown anaerobically from an initial OD₆₀₀ of 0.05 in MM at 25° C. Irradiating CdSe-MPA (1 μM) in the culture with 530-nm light over the course of one week (168 hours) resulted in 18.2 μmol±5.2 μmol H₂. In the absence of CdSe-MPA, WT produced 6.2 μmol (±3.2 μmol) of H₂ by the end of one week (FIG. 2). MR-1 has periplasmic [Ni—Fe] and [Fe—Fe] hydrogenases (H₂ase) that may reduce protons during anaerobic respiration. The H₂ produced by WT is consistent with this activity. In the CdSe-MPA QD mixtures containing WT, most H₂ evolution activity was observed within the first 120 hours, plateauing by the end of one week. During the fastest period of H₂ evolution (between 12 to 72 hours), a rate of 2.6×10⁻¹ μmol/hr was observed, an order of magnitude faster than WT in MM achieved in the same time frame.

FIG. 8 shows the rate of hydrogen evolution from WT and CdSe-MPA. The most linear portion of the kinetics of hydrogen evolution, between 12 and 72 hours, were used to gauge the rates of hydrogen evolution from WT MR-1 with and without CdSe-MPA. Representative reactions are shown for vials containing WT (initial OD₆₀₀ of 0.05) and CdSe-MPA (1 μM, as shown) in minimal medium (MM), irradiated with green LEDs (530 nm) at 25° C. The observed rates result in averages of 2.6×10⁻¹ μmol/hr for the system containing CdSe-MPA, and 1.6×10⁻² μmol/hr for systems without CdSe-MPA.

### WT Supports CdSe-MPA Photocatalysis

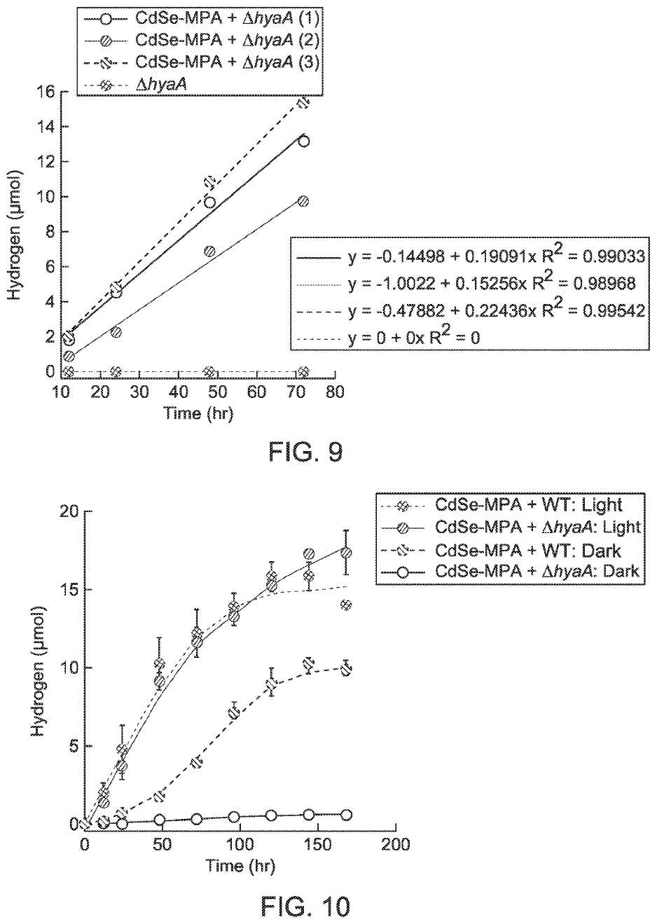

The increased H₂ evolution activity of CdSe-MPA in the presence of WT is consistent with WT supporting CdSe-MPA photocatalysis, but the background H₂ evolution activity of WT is a complicating factor. To test the hypothesis that the enhanced H₂ production in the complete system results from bacterial respiration of CdSe-MPA, rather than an increase of MR-1 H₂ production activity in the presence of CdSe-MPA, the MR-1 gene knockout hyaA (ΔhyaA) was used. The hyaA gene encodes the small subunit of periplasmic [Ni—Fe]-H₂ase. Consistent with impaired hydrogenase activity, ΔhyaA produced no detectable H₂ over the course of one week when cultured in MM. However, irradiation of CdSe-MPA within a culture of ΔhyaA results in H₂ evolution (FIG. 2). The total H₂ evolved from CdSe-MPA and ΔhyaA was 20.8 μmol±5.8 μmol H₂ after one week, which is similar to the activity of CdSe-MPA in the presence of WT (FIG. 2). The overall rate of H₂ evolution is similar for CdSe-MPA with WT or ΔhyaA, with the highest rate achieved (1.6×10⁻¹ μmol/hr) on the same order of magnitude as the system containing CdSe-MPA and WT, slowing after approximately 120 hours (FIG. 9, FIG. 2).

FIG. 2 shows hydrogen evolution from CdSe-MPA (1 μM) with WT (initial OD₆₀₀=0.05, as labelled) and ΔhyaA MR-1 (initial OD₆₀₀=0.05, as labelled), monitored under constant irradiation (530 nm) at 25° C., pH 7. Hydrogen evolution in the absence of CdSe-MPA was also recorded. FIG. 2A) shows the total H₂ evolved after one week (168 hours). Error bars represent the standard deviation from ten replicate experiments. FIG. 2 B) shows H₂ monitored over time (0 to 168 hours). Error bars represent the range of values observed at each time point for triplicate experiments.

FIG. 9 shows the rate of hydrogen evolution from ΔhyaA and CdSe-MPA. The most linear portion of the kinetics of hydrogen evolution, between 12 and 72 hours, were used to gauge the rates of hydrogen evolution from ΔhyaA with and without CdSe-MPA. Representative reactions are shown for vials containing ΔhyaA (initial OD₆₀₀ of 0.05) and CdSe-MPA (1 μM, as shown) in modified minimal medium (MM), irradiated with green LEDs (530 nm) at 25° C. The observed rates result in averages of 2.6×10⁻¹ μmol/hr for the system containing CdSe-MPA, and 1.6×10⁻² μmol/hr for systems without CdSe-MPA.

The production of H₂ in the system using ΔhyaA supports the hypothesis that MR-1 is providing electrons to CdSe-MPA through respiration.

QDs Act as Photocatalysts with Activity Sustained by Bacterial Respiration

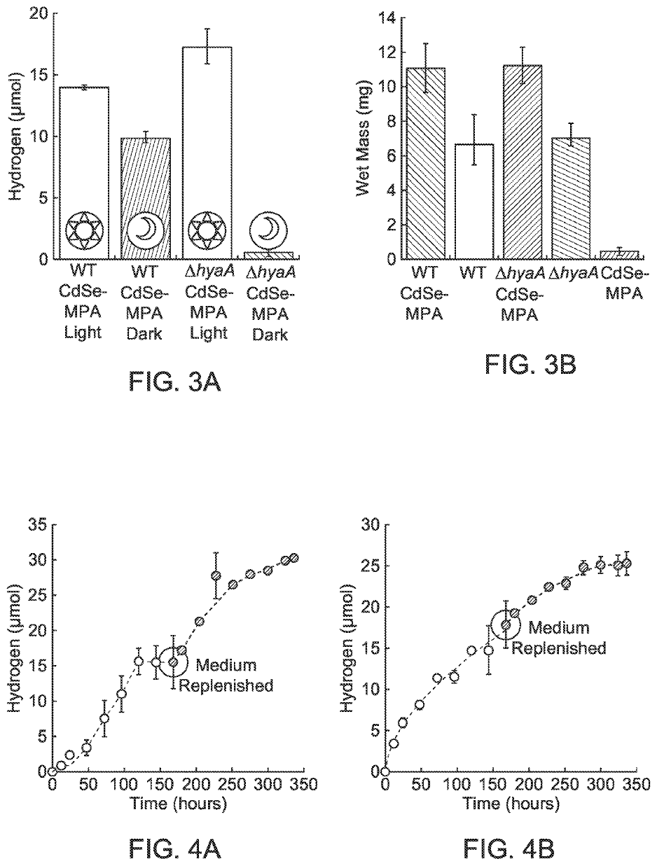

With CdSe-MPA present in a WT culture, H₂ evolution was monitored in the light and dark over the course of one week (FIG. 10). In the dark, 4.1 μmol less H₂ on average was produced compared to samples grown in the light (FIG. 3). The amount of H₂ produced in the dark by CdSe-MPA and WT (9.8 μmol±0.6 μmol) is similar to the amount of H₂ produced by WT without QDs present. The dependence of activity on irradiation was tested with CdSe-MPA in cultures of ΔhyaA, monitoring the system's H₂ evolution in the light and dark (FIG. 10). FIG. S6 shows hydrogen evolution from WT (initial OD₆₀₀ of 0.05, as labelled) and ΔhyaA (initial OD₆₀₀ of 0.05, as labelled) with CdSe-MPA (1 μM) monitored in the light (green LEDs (530 nm)) and dark over the course of one week at 25° C.

A mixture of CdSe-MPA and ΔhyaA in the dark resulted in negligible H₂ evolution (<1 μmol), whereas in the light 17.4+1.4 μmol H₂ were produced (FIG. 3). The light-dependent evolution of H₂ supports the hypothesis that the QDs act as photocatalysts with activity sustained by bacterial respiration.

In the presence of exogenous electron acceptors, the growth of MR-1 is altered. After one week of catalysis, vials containing CdSe-MPA paired with WT or ΔhyaA were agitated to create a homogeneous solution. The solutions were centrifuged and the total wet mass of the resulting pellets measured. The same procedure was performed on solutions of WT or ΔhyaA cultured in the absence of QDs for one week. Systems containing CdSe-MPA showed higher wet mass, indicating more bacterial growth, than those without (FIG. 3).

FIG. 3A) shows total H₂ evolution from WT (initial OD₆₀₀ 0.05) and ΔhyaA MR-1 (initial OD₆₀₀=0.05) with CdSe-MPA (1 μM) as monitored in the light (530 nm) and dark after one week at 25° C., pH 7. Error bars represent the range observed in triplicate experiments. FIG. 3 B) shows accumulated mass after a week-long catalysis with WT (initial OD₆₀₀=0.05) and ΔhyaA MR-1 (initial OD₆₀₀=0.05) with and without CdSe-MPA (1 μM) (method of measurement described in FIG. 10.) With CdSe-MPA, total masses using WT and ΔhyaA were 11.1 mg (±1.4 mg) and 11.3 mg (±1.1 mg), respectively. Without CdSe-MPA, the respective wet masses for WT and ΔhyaA were 6.7 mg (±1.3 mg) and 7.0 mg (±0.8 mg). The differences in overall mass are not accounted for by CdSe-MPA alone (0.5 mg±0.2 mg). Other studies have shown that CdSe NCs exhibit toxicity to MR-1; however, in catalytic concentrations reported here (1 μM, versus >200 μM in toxicity studies), CdSe-MPA increase the total biomass formation of MR-1, suggesting that MR-1 respires CdSe-MPA.

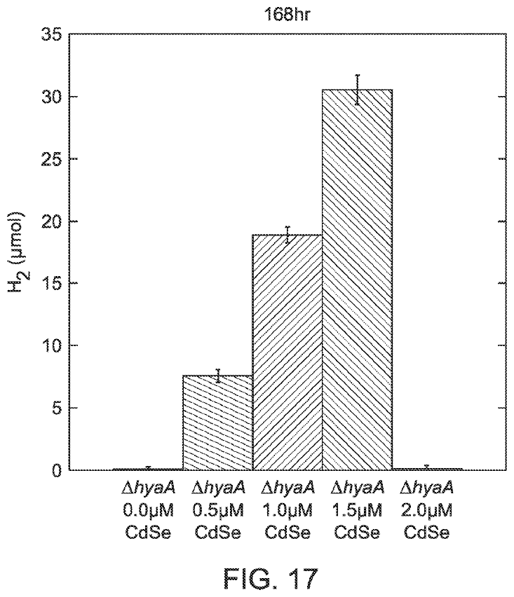

To determine the effect of CdSe-MPA concentration on hydrogen production, CdSe-MPA at concentrations 0.0, 0.5, 1.0, 1.5, and 2.0 UM were added to a culture of Shewanella ΔhyaA and the yield of hydrogen measured after 168 hours. Hydrogen production increased linearly up to 1.5 μM CdSe, and then decreased (FIG. 17).

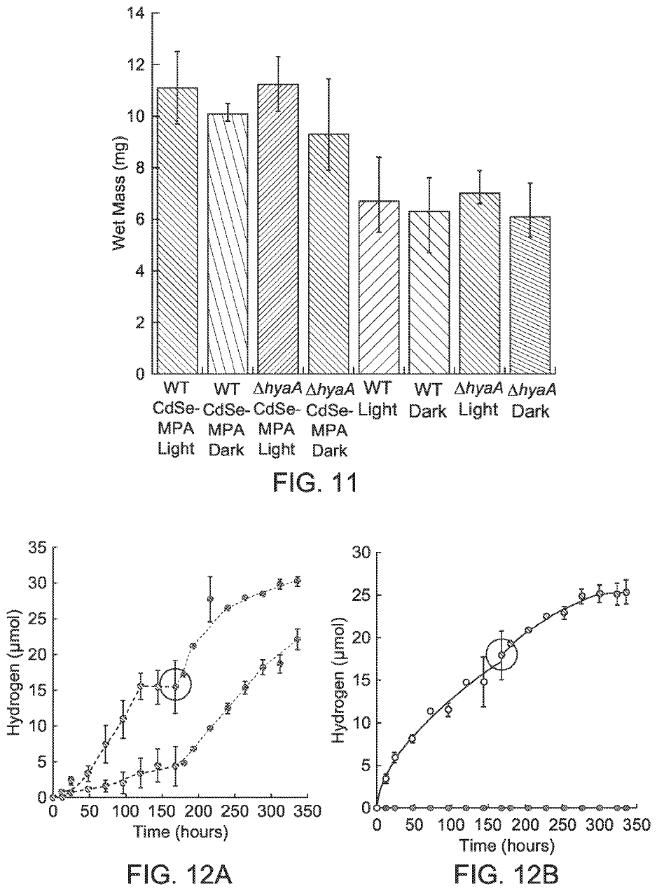

Although greater total H₂ evolution was only observed in the light, increased bacterial growth in the presence of CdSe-MPA was observed in both light and dark conditions. FIG. 11 shows the breakdown of mass accumulated after all conditions in the light and dark, which demonstrates that the same trends are observed in the dark as the light.

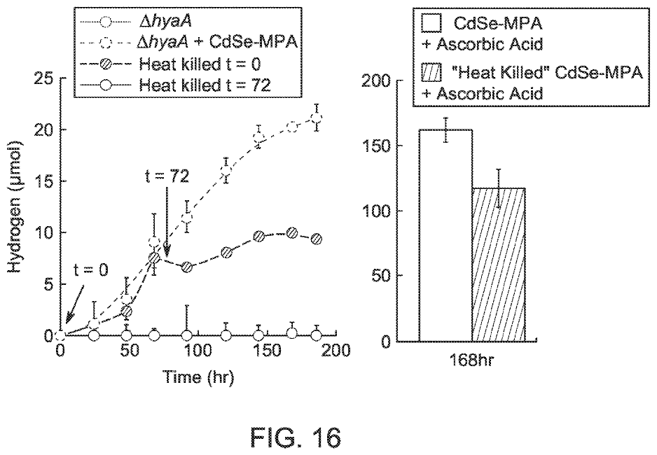

To demonstrate that the activity of live cells is responsible for the functioning of the system, bacteria were heat killed to test the effect on the system using Shewanella ΔhyaA which has no background hydrogen production from the bacteria (FIG. 16). If the combination of CdSe-MPA and Shewanella ΔhyaA in minimal medium is heated to 100° C. and the system is irradiated with 530-nm LEDs, no hydrogen is produced. Furthermore, if the combination of CdSe-MPA and Shewanella ΔhyaA in minimal medium is allowed to grow for 72 hours and then heated to 100° C. and irradiated with 530-nm LEDs, hydrogen production plateaus 72 hours. However, heating CdSe-MPA and a chemical electron donor, ascorbic acid, has a smaller impact on system activity upon 530-nm LED irradiation. This result further demonstrates that, without functioning biological electron transfer pathways from living Shewanella ΔhyaA, no hydrogen evolution activity is observed. The production of H₂ in the system using ΔhyaA supports the hypothesis that MR-1 is providing electrons to CdSe-MPA through respiration.

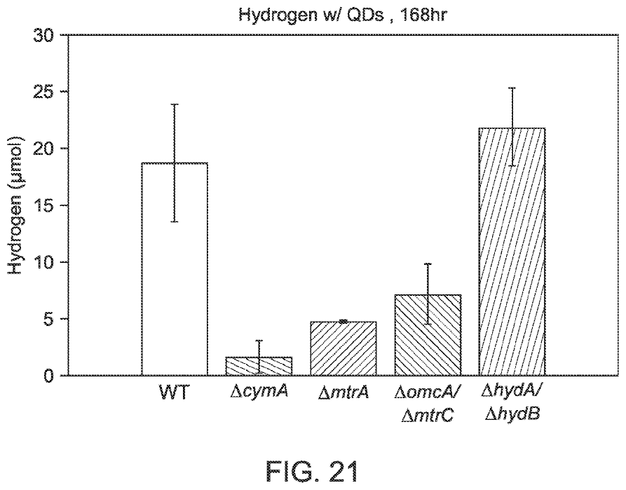

To demonstrate that extracellular electron transfer from MR-1 supports hydrogen production by CdSe-MPA, strains of MR-1 with genes associated with extracellular electron transfer knocked out were used in photochemical experiments (strains: ΔcymA, ΔmtrA, ΔomcA/ΔmtrC). In comparison with wild-type (WT) MR-1 and the ΔhydA/ΔhyaB double knockout incapable of hydrogenase expression, systems with the extracellular electron transfer genes knocked out showed significantly lower hydrogen production (FIG. 21). This result supports the hypothesis that extracellular electron transfer from MR-1 supports hydrogen production by CdSe-MPA.

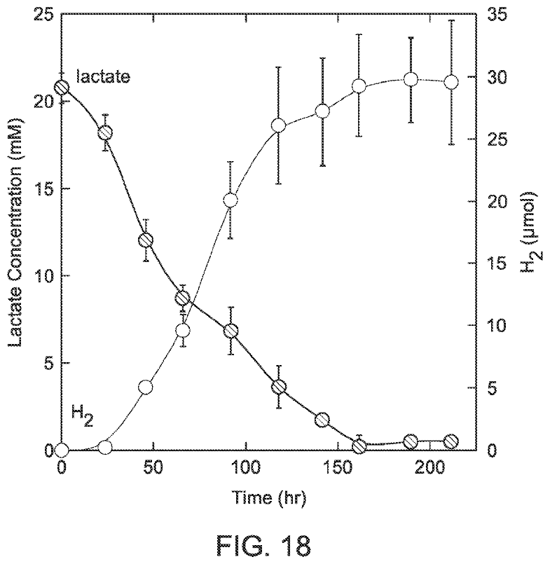

H₂ evolution activity in MR-1: CdSe-MPA systems plateaus after approximately one week. MR-1 utilizes lactate in the growth medium as a primary carbon source. We tracked both the consumption of lactate and the evolution of hydrogen over time using Shewanella ΔhyaA (FIG. 18). We determined an initial lactate concentration of 20.8±0.9 mM. After 212 hours of reaction with irradiation at 530 nm, the lactate concentration was depleted to 0.6±0.2 mM. Hydrogen production was also measured over this time and increased from 0 to 29 mmol. Notably, lactate concentration and H₂ production over time are anticorrelated, with both leveling off after approximately 150 hours. This result supports the hypothesis that lactate is the terminal electron source for this system, and that system activity is limited by lactate availability.

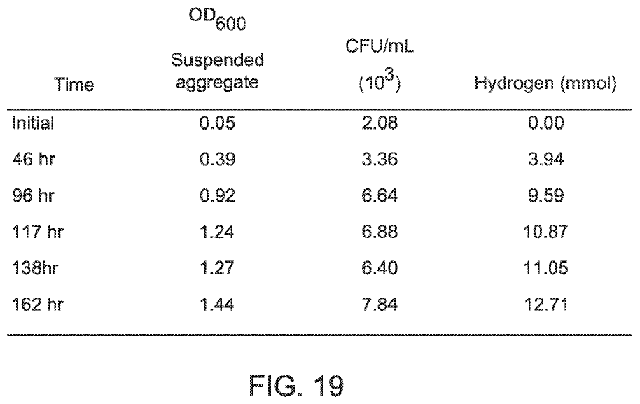

To demonstrate cell viability, we performed colony forming units (CFU) analysis over time in a MR-1: CdSe-MPA system upon irradiation, while also measuring H₂ production. CFU data were obtained on the aggregate formed between CdSe-MPA and Shewanella in the photoreactor vials. The results (FIG. 19) show that CFU increases over time, demonstrating the presence of viable bacteria over the experiment.

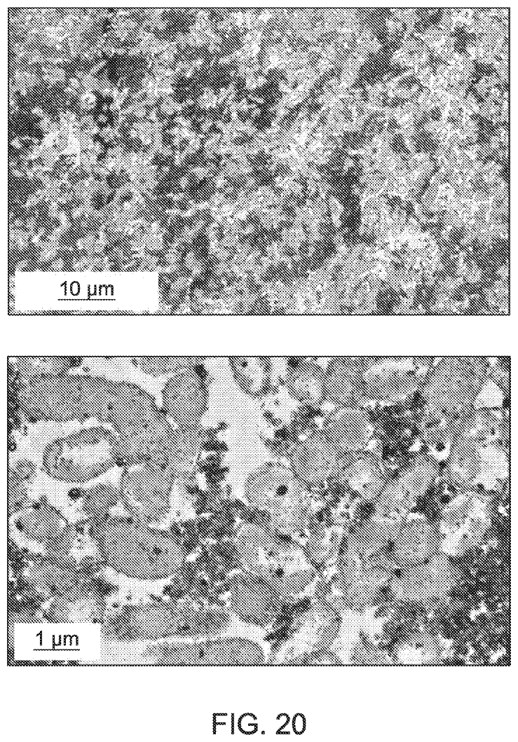

To visualize the aggregate formed between Shewanella and CdSe-MPA, electron microscopy was performed. The electron microscopy images of the aggregate reveal bacteria surrounded by QDs, with contacts between QDs and the outer membrane (FIG. 20).

Carbon dots (CDs) Impact the Growth of MR-1

As noted in other studies, water-soluble carbon nanomaterials, carbon dots (CDs), were shown to impact the growth of MR-1. The presence of CDs was found to boost the metabolic rate of MR-1, lead to increased biofilm formation, and possibly increased intercellular signaling. Interactions between MR-1 and nanoparticulate TiO₂ also promoted increased riboflavin secretion, possibly boosting the electrogenic behavior of MR-1. The interaction between MR-1 and nanomaterials is complex, varying by nanomaterial concentration, composition, and charge. Given the enhanced bacterial growth with CdSe-MPA observed in both the light and the dark, it is possible that the presence of the QDs could promote biofilm formation. Consistent with biofilm formation, bacteria and QDs formed aggregates (MR-1: CdSe-MPA) during experiments.

As noted before, H₂ evolution activity in MR-1: CdSe-MPA systems plateaued after approximately one week. Bacteria-QD aggregates were separated from MM containing planktonic cells and colloidal CdSe-MPA after one week, and fresh MM was added to the aggregates to a total amount of 5.0 mL in each vial. Upon replenishing the medium and irradiating the mixtures, H₂ evolution activity resumed. FIG. 4 shows hydrogen produced by the MR-1: CdSe-MPA system in MM, with fresh MM added to A) CdSe-MPA (1 μM) with WT (initial OD₆₀₀=0.05, as labelled) and B) ΔhyaA, MR-1 (initial OD₆₀₀=0.05, as labelled) after 168 hours. Hydrogen evolution was monitored for an additional 168 hours under constant irradiation (530 nm) at 25° C., pH 7. Error bars represent the range of values observed at each time point for triplicate experiments. In many cases, error bars are small relative to the data point.

Combined with WT, CdSe-MPA sustained 95% of the original activity (measured in total H₂ evolved) over the second week. When combined with ΔhyaA, 41% of the original activity was recovered. After replenishing the medium, MR-1: CdSe-MPA aggregates reform within 12 hours. In the absence of CdSe-MPA during the second week, WT in the presence of fresh MM resumed evolving H₂, while ΔhyaA produce no H₂ over the course of two weeks. FIG. 12 shows hydrogen evolution after replenishing MM with and without CdSe-MPA. After 168 hours, fresh MM was added to A) CdSe-MPA (1 μM) with WT (initial) (orange) and WT (initial OD₆₀₀ of 0.05) in the absence of CdSe-MPA (gray). B) Fresh MM was added to CdSe-MPA (1 μM) and ΔhyaA MR-1 (initial OD₆₀₀ of 0.05) (purple) and in the absence of CdSe-MPA (gray). Hydrogen evolution was monitored for an additional 168 hours under constant irradiation (530 nm) at 25° C., pH 7 These results suggest that CdSe-MPA retain their activity after a week of catalysis, and that the bacteria continue to provide electrons to CdSe-MPA within the reformed aggregates.

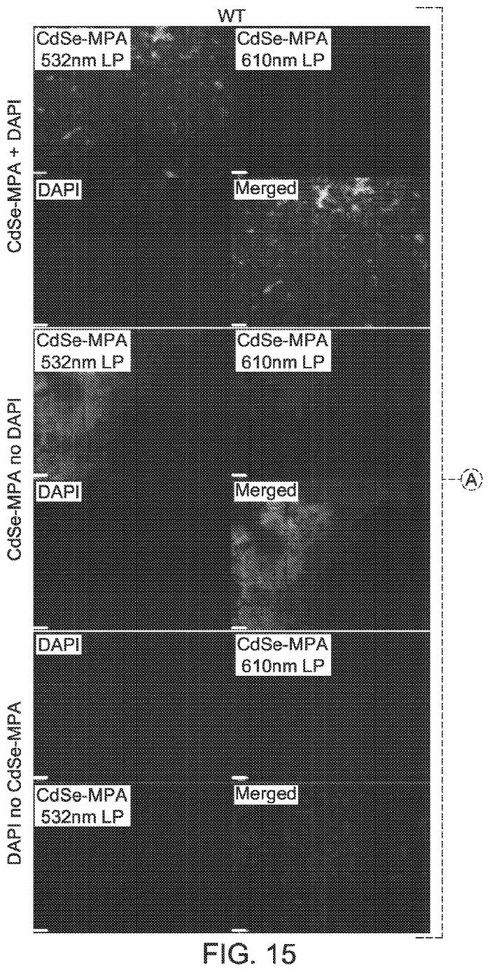

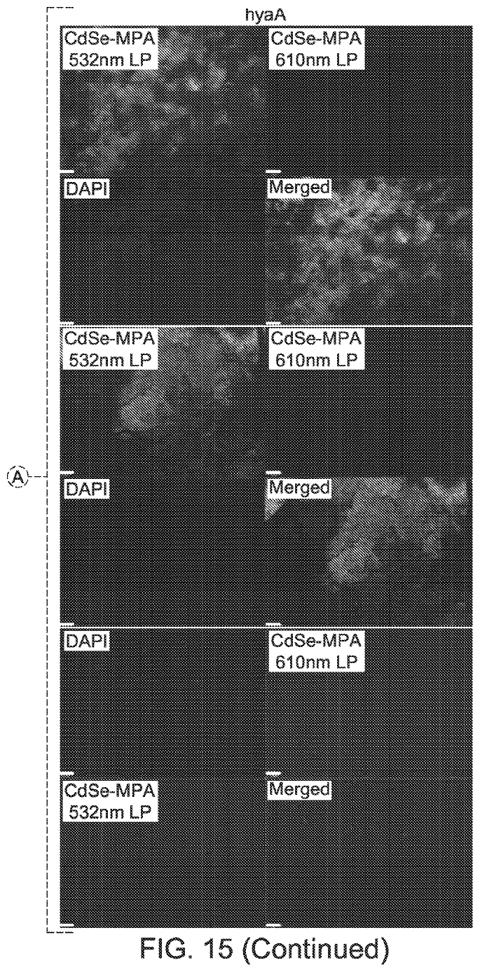

The MR-1: CdSe-MPA aggregates retain the bright orange color of CdSe-MPA. This is also true for cell pellets formed by centrifugation post-experiment, where cells in systems containing CdSe-MPA retain the orange coloration of the QDs. The presence of both MR-1 and CdSe-MPA in the MR-1: CdSe-MPA aggregate was confirmed with fluorescence microscopy. FIG. 15 are fluorescence microscopy images demonstrating the presence of CdSe-MPA and MR-1 in MR-1: CdSe-MPA aggregates. Excitation at 350 nm was carried out on slides containing WT or ΔhyaA and CdSe-MPA aggregates stained with DAPI (top, as labelled). A band pass (BP) filter of 450 nm (“DAPI”) shows the stained MR-1. The same image was collected using an excitation line/emission LP filter pair of 405 nm/532 LP to collect luminescence specific to emission from CdSe-MPA. Extending the LP filter to 610 nm, outside of the expected photoluminescence range for CdSe-MPA, negates the observed luminescence. Control slides with no DAPI staining (center, labelled) show luminescence only from CdSe-MPA. Control slides stained with DAPI but lacking CdSe-MPA show no luminescence with the LP filter of 532 nm, confirming the observed PL is from CdSe-MPA rather than an unknown fluorophore in MR-1.

Thiol-coated CdSe NCs are reported to precipitate due to photocatalytic oxidation of the thiol ligands as well as photooxidation of the NCs. This is supported by the observation of a red-brown precipitated solid for CdSe-MPA held under irradiation in MM in the absence of bacteria, which precipitate within 72 hours. To test if the precipitated CdSe-MPA were still catalytically active, ΔhyaA were added to a solution of precipitated QDs in MM.

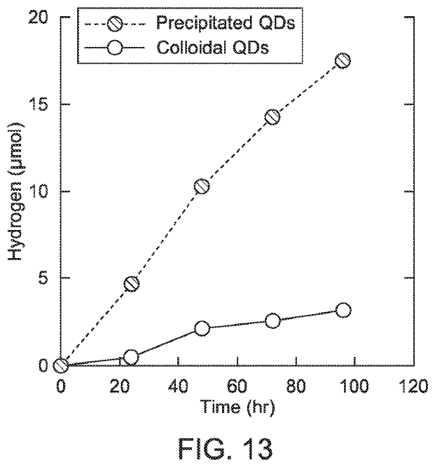

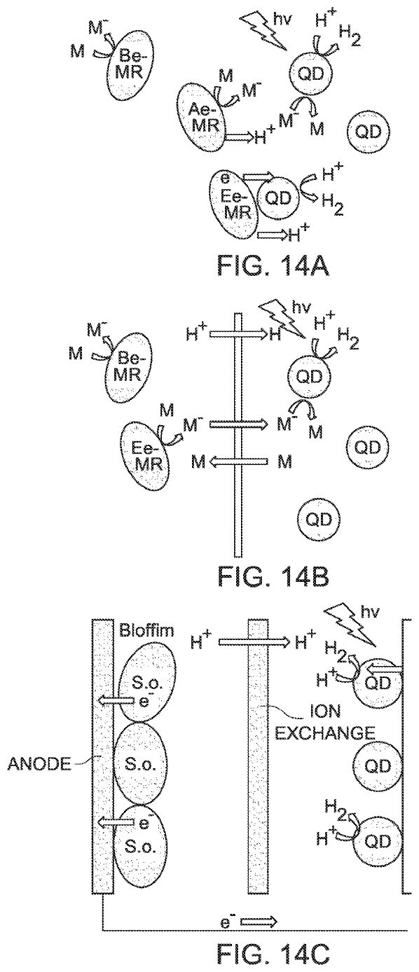

Hydrogen evolution from precipitated CdSe-MPA after ΔhyaA is introduced begins to plateau after only 48 hours (FIG. 13). The total H₂ evolution from precipitated CdSe-MPA in the presence of ΔhyaA was minimal, achieving only 18% of the total H₂ evolved from colloidal CdSe-MPA with ΔhyaA. The decrease in activity is likely due to photooxidation of the QDs. The interaction between CdSe-MPA and bacteria may slow down the photooxidation of CdSe-MPA, helping to sustain activity for over two weeks. Previously, TONs per QD (TONQD) for photocatalytic H₂ evolution from CdSe-MPA have ranged from 10,000 to 20,000, with external QYs of approximately 1.5% to 9.5% at an acidic pH of 4.5. Sustained by bacteria, a QY of 0.2% is achieved here, along with a TONQD of 4160 at neutral pH (Table 9 for further details). CdSe-MPA have been used in H₂ evolution systems in which electrons are provided by a large excess of ascorbic acid (>500 mM). Here, MR-1 thrives with a relatively low (20 mM) concentration of lactate as a primary carbon source. Notably, lactate is abundant in wastewater, present in a variety of food processing industrial liquid waste, and also as a product of fermentation. Ranging from approximately 3 mM to upwards of 100 mM, lactate is already present in these wastewater sources at a concentration necessary to sustain MR-1 growth. In addition, assuming that the complete oxidation of lactate yields electrons that go to H₂ formation in the CdSe-MPA MR-1 cultures used herein, it would be possible to produce 100 μmol of H₂ per each 5 mL solution (100 μmol lactate). Based on this metric, H₂ evolution fueled by MR-1 results in a 20.8% yield with respect to lactate. Using the typical sacrificial electron donor ascorbate for photocatalytic H₂ production by CdSe-MPA creates only a 6.8% yield. These initial TONQD, yield, and QY values from CdSe-MPA sustained by MR-1 are obtained without optimization of the system. Further optimizing hole transfer to MR-1 will enable an even more active QD system.

The above example demonstrates a fully light-driven system that utilizes the natural respiratory pathway of an electrogenic bacterium, Shewanella oneidensis MR-1, to provide electrons to QD photocatalysts (in this embodiment CdSe-MPA QDs) for H₂ evolution. MR-1 uses lactate, an industrial waste product, as an energy source to sustain growth and H₂ evolution for over a week. The growth of MR-1 is not diminished by QDs in catalytic concentrations, but rather, their growth is enhanced. A major advantage of a nanocrystalline photocatalysis system driven by electrogenic bacteria is its design flexibility, as the combination of different electrogenic bacteria and various NCs may also enhance electron transfer and film formation. Possible routes of improvement could be focused on genetically engineering bacteria and/or tailoring NCs. Adding light-harvesting components with the ability to absorb a greater portion of the solar flux may also improve quantum yields. In addition, NCs can be tailored to carry out a variety of catalytic reactions. Catalytic cascades taking advantage of both NC photocatalysis and reactions carried out by microbes also can be envisioned. The robust nature of the H₂ evolution system described herein demonstrates the expanding possibilities at the interface of bio- and nano-technology.

### Materials and Methods

### Cell Culturing Conditions

Prior to experiments, both Shewanella oneidensis MR-1 wild-type (WT) strain and knockout mutant ΔhyaA were plated on LB agar plates and incubated for 24 hours at 30° C. A colony from each strain was transported to 50 mL LB medium, where cultures of ΔhyaA contained

$$30 \frac{μg}{mL}$$

kanamycin. The cultures were grown anaerobically at 30° C., with shaking at 100 rpm to an OD₆₀₀ of 0.4-0.5 after ~18 hours. At the target OD₆₀₀, 100 μL of each strain were transferred to sealed vials containing 6 mL of modified minimal medium (MM), with an additional

$$30 \frac{μg}{mL}$$

kanamycin in vials growing ΔhyaA. Casamino acids (Gibco, Thermo Fischer) were dissolved in DI water and filtered to create a 10% stock solution which was immediately transferred to the 6 mL MM vials, equaling 1% total concentration in the culture. In these vials, the cultures were allowed to grow to an OD₆₀₀ of 0.4-0.5 for 24 hours at 30° C. with shaking at 100 rpm to be transferred for use in photochemical experiments.

### Minimal Medium Composition and Preparation

The solutions listed in tables 1~4 were each prepared separately. Each solution component of the minimal medium composition (MM) was adjusted to pH 7.0 using NaOH or HCl while being protected from light. The amino acid solution was made and stored at +4° C. for a maximum of 15 minutes before being added to the MM. The solution components were sealed and mixed anaerobically with N₂ gas in the proportions given, and subsequently autoclaved at 120° C. for 2 hours. MM was prepared with 30 mM fumarate as an electron acceptor for bacteria grown prior to photochemical experiments, and with no fumarate for MM used in photochemical experiments.

TABLE 1
Minimal medium recipe. Minimal medium was prepared
according to the recipe shown in Table 1.

| | | | | | | Final |
|---|---|---|---|---|---|---|
| | | | | | Vendor/ | conc. in |
| Chemical description | FW | g/L | | Formula | Cat # | MM, nM |
| PIPES buffer | 302.4 | 15.16 | | C₈H₁₈N₂O₆S₂ | Sigma P1851 | 50 |
| Sodium hydroxide | 40.00 | 0.3 | | NaOH | Sigma S5881 | 7.5 |
| Ammonium chloride | 53.49 | 1.5 | | NH₄Cl | Sigma A5666 | 28.04 |
| Potassium chloride | 74.55 | 0.1 | | KCl | Sigma P4504 | 1.34 |
| Sodium phosphate | 138.0 | 0.6 | | NaH₂PO₄•H₂O | Sigma S9638 | 4.35 |
| monobasic | | | | | | |
| Sodium chloride | 58.44 | 1.75 | | NaCl | Fisher S271-3 | 30 |
| Minerals solution | | 10 | mL | | | |
| 100X stock | | | | | | |
| Vitamins solution | | 100 | μL | | | |
| 10000X | | | | | | |
| Lactic acid | 90.08 | 1.66 | mL | CH₃CH(OH)CO₂H | | 20 |
| Amino acid | | 10 | mL | | | |
| solution 100X | | | | | | |

TABLE 2
Vitamin solution 10,000X. The vitamin solution used in the minimal medium
(Table 1) was prepared according to the recipe shown in Table 2.

| | | | | | Final |
|---|---|---|---|---|---|
| | | g/100 mL | | Vendor/ | conc. in |
| Chemical description | FW | (10000X) | Formula | Cat # | MM, nM |
| Biotin (d-biotin) | 244.3 | 0.02 | C₁₀H₁₆N₂O₃S | Sigma B4639 | 81.87 |
| Folic acid | 441.1 | 0.02 | C₁₉H₁₉N₇O₆ | Sigma F7876 | 45.34 |
| Pyridoxal HCl | 205.6 | 0.1 | C₈H₁₂ClNO₃ | Sigma P9130 | 486.38 |
| Riboflavin | 376.4 | 0.05 | C₁₇H₂ON₄O₆ | Sigma R4500 | 132.84 |
| Thiamine HCl 1.0 H2O | 355.3 | 0.05 | C₁₈H₁₈Cl₂N₄OS | Sigma T4625 | 140.73 |
| Nicotinic acid | 123.1 | 0.05 | C₆H₅NO₂ | Sigma N4126 | 406.17 |
| D-pantothenic acid, | 238.3 | 0.05 | C₉H₁₆NO₅•½Ca | Sigma P2250 | 209.82 |
| hemicalcium salt | | | | | |
| Vitamin B12 | 1355.4 | 0.001 | C₆₃H₈₈CoN₁₄O₁₄P | Sigma V2876 | 0.74 |
| 4-aminobenzoic acid | 137.13 | 0.05 | C₇H₇NO₂ | Sigma A9878 | 364.62 |
| Thioctic acid | 206.3 | 0.05 | C₈H₁₄O₂S₂ | Sigma T5625 | 242.37 |
| (α-lipoic acid) | | | | | |

TABLE S
Mineral solution 100X. The mineral solution used in the minimal medium
(Table 1) was prepared according to the recipe shown in Table 3.

| | | | | | Final |
|---|---|---|---|---|---|
| | | g/L | | Vendor/ | conc. in |
| Chemical description | FW | (100X) | Formula | Cat # | MM, μM |
| Nitrilotriacetic acid (dissolve | 191.1 | 1.5 | C₆H₉NO₃ | Sigma N9877 | 78.49 |
| with NaOH to pH 8) | | | | | |
| Magnesium sulfate heptahydrate | 246.48 | 3 | MgSO₄•7H₂O | Aldrich 23039-1 | 121.71 |
| Manganese sulfate monohydrate | 169.02 | 0.5 | MnSO₄•H₂O | Aldrich 22,128-7 | 29.58 |
| Sodium chloride | 58.44 | 1 | NaCl | Sigma S3014 | 171.12 |
| Ferrous sulfate heptahydrate | 277.91 | 0.1 | FeSO₄•7H₂O | Sigma F8633 | 3.60 |
| Calcium chloride dehydrate | 146.99 | 0.1 | CaCl₂•2H₂O | Sigma C3881 | 6.80 |
| Cobalt chloride hexahydrate | 237.93 | 0.12 | CoCl₂•6H₂O | Sigma C3169 | 5.20 |
| Zinc chloride | 136.28 | 0.13 | ZnCl₂ | Sigma Z3500 | 9.54 |
| Cupric sulfate pentahydrate | 249.68 | 0.01 | CuSO₄•5H₂O | Sigma C6283 | 0.40 |
| Aluminum potassium | 474.38 | 0.01 | AlK(SO₄)2•12H₂O | Sigma A7176 | 0.21 |
| disulfate dodecahydrate | | | | | |
| Boric acid | 61.83 | 0.01 | H₃BO₃ | Sigma B6768 | 1.62 |
| Sodium molybdate dehydrate | 241.95 | 0.025 | Na₂MoO₄•2H₂O | Aldrich 22,184-8 | 1.03 |
| Nickel chloride hexahydrate | 237.6 | 0.024 | NiCl₂•6H₂O | Sigma N6136 | 1.01 |
| Sodium tungstate | 329.86 | 0.025 | Na₂WO₄•2H₂O | Sigma S0765 | 0.76 |

TABLE 4
Amino acid solution 100X. The amino acid solution
used in the minimal medium (Table 1) was prepared
according to the recipe shown in Table S4.

| Chemical | g/100 mL | | Final conc. in |
|---|---|---|---|
| description | (100X) | Vendor/Cat # | MM, mg/mL |
| L-glutamic acid | 0.2 | Sigma G1251 | 2 |
| L-arginine | 0.2 | Sigma A3909 | 2 |
| D- or L-serine | 0.2 | Sigma S4375 | 2 |

### Synthesis of CdSe-MPA

Inside a nitrogen filled glovebox, 0.79 g of Selenium was dissolved in 10 mL of 97% trioctylphosphine in a scintillation vial and allowed to stir at 50° C. until all solid had dissolved. In a separate vial, 0.747 g of anhydrous Cadmium Acetate was dissolved in 12 ml of 97% trioctylphosphine and stirred at 50° C. until all solid had dissolved. Outside of the glovebox, to a 100 mL three neck flask was added 7.8 g of trioctylphosphine oxide, 2.3 g of 98% hexadecylamine, and 0.171 g of 97% tetradecylphosphonic acid. The flask was purged with N₂ three times and heated to 100° C. below 0.1 Torr with stirring for 30 minutes. After returning to N₂, 2.5 mL of the TOP/Se solution was injected, and the reaction was heated to 310° C., where 2.5 mL of the TOP/Cd solution was injected very quickly with a syringe. The flask was transferred to a heating mantle set to 260° C., and the reaction was allowed to stir for 7 minutes. The solution was cooled using a heat gun (set to low) and placed into a water bath. 15 mL of hexanes was injected into the reaction to prevent solidification of TOPO. The mixture was separated into 2 50-mL centrifuge tubes, where 10 ml of methanol and 25 mL of acetone was added. The product was centrifuged for 15 minutes at 8000 RPM. The clear supernatant was discarded, and the solid pellet was allowed to air dry. The solid was dispersed in 10 mL hexanes and placed in the centrifuge again for 15 minutes at 8000 RPM (repeated as needed). The final product was dissolved in hexanes.

To exchange the native ligands with mercaptopropionic acid (MPA), 0.2401 g of tetrame-thylammonium hydroxide pentahydrate was dissolved in 13.3 mL of methanol along with 82.7 μL of MPA. 100 nmol of TOPO-CdSe QDs in hexanes was added to 7.33 mL of the methanol solution in a 100 mL round bottom flask. The flask was put under N₂ gas and refluxed at 65 C for 45 minutes. The solution was then dived into 2 centrifuge tubes, where 35 mL of ethyl ether and 10 mL of ethyl acetate were added. The tubes were placed into a centrifuge and spun at 8000 RPM for 15 minutes. The supernatant was disposed of, and the pallet was allowed to air dry. The results solid was dispersed in water.

### Photochemical Experiments

Prior to beginning photochemical experiments, 41-mL scintillation vials (Sigma-Aldrich) and custom gas-tight caps containing GC septa (Restek) were autoclaved. Each experimental solution was a total of 5.0 mL in modified minimal medium (MM, pH 7). After bacterial cultures were grown to an OD₆₀₀ of 0.4-0.5, approximately 500 μL of the cultures were transferred to MM (no fumarate) to reach an OD₆₀₀ of 0.05 in photochemical vials. CdSe-MPA were added to a final concentration of 1 μM, typically requiring about 230 μL from a CdSe-MPA stock solution. Each 5-mL solution in a 41-mL vial was sealed with a gas-tight cap and a septum and deoxygenated by purging the solution for 20 min and headspace for 10 min with a 79.31%/20.69% N₂/CH₄ (Airgas) mixture using sterile needles. CH₄ is present as a reference for product quantification.

Irradiation in the photochemical experiments was performed in a custom-built 16-sample apparatus where 41-mL scintillation vials were placed. The samples are temperature controlled at 25° C. with a circulating water bath, and maintain constant shaking at 100 rpm throughout the experiment. Samples are illuminated from below by a light-emitting diode (Philips LumiLED Luxeon Star Hex green 700 mA LEDs) at 530 nm (±10 nm). At the beginning of each experiment, the power of each LED was set to 25 mW±5 mW, as measured with a Nova II power meter (Ophir-Spiricon LLC) placed over each of the 16-wells. The amount of H₂ produced was determined by sampling the headspace via gas chromatography (GC). To monitor H₂ evolution, 25 μL of headspace gas was withdrawn from each vial through a septum with a 50 μL gas-tight syringe (Hamilton). Headspace gas samples were analyzed on a Shimadzu GC-2014AT gas chromatograph (GC) with a thermal conductivity detector and Carboxen 1010 PLOT column (30 m×0.53 mm, Supelco) to quantify the H₂ evolved with reference to CH₄ (in the purging gas mixture) as the internal standard.

### Preparation of Samples for Microscopy

A glass coverslip (Fisherbrand 18×18-1, cat. 12542A) was cut in half and inserted into the bottom of 41 mL experimental vials and autoclaved prior to experimental setup. The glass coverslip was used to remove the bacteria: QD aggregates without disturbing their architecture post-experiment. As described above, experiments combining WT and ΔhyaA MR-1 with CdSe-MPA were carried out for one week, and MR-1: CdSe-MPA aggregate formation was observed. After a week-long experiment, the supernatant was carefully removed from around the aggregates, which adhered to the coverslip as liquid was removed. To ease the process of removing the coverslip from the bottom of the vial without disturbing the aggregate, a low flow of nitrogen through a sterile air filter was used to dry the coverslip while it remained in the bottom of the vial. After drying for 45 minutes, a sterile inoculation loop was used to displace the coverslip, and the coverslip was then removed with sterile tweezers and placed onto clean slides. To prepare slides of the WT and ΔhyaA MR-1 in the absence of QDs (no aggregate formation), planktonic cells were transferred from media by placing two drops of media onto a clean microscope slide. They were then left to dry in sterile petri dishes under a low flow of sterile nitrogen for approximately one hour.

Slides were washed with a few drops of PBS, pH 7.4, prior to fixation. Fixation of cells was carried out using 10% formalin (equivalent 4% paraformaldehyde) in PBS at pH 7.4 for 10 min at room temperature. After fixation, cells were gently washed with PBS. For membrane permeabilization to allow entry of DAPI, Triton X-100 was used. A 0.05% v/v Triton solution was prepared, and cells were incubated with Triton for 3 min at room temperature. Cells were rinsed with PBS immediately after permeabilization to remove the detergent. A DAPI stock solution was prepared at 20 mg/mL in DI H₂O from lyophilized DAPI powder. A diluted stock of 0.5 μg/mL was prepared in PBS for staining the cells. 500 μL of DAPI (0.5 μg/mL) were added to each slide, which incubated for 5 minutes at room temperature in the dark. After staining, cells were rinsed with PBS. For slides containing a coverslip and aggregate, they were secured to the microscope with a minimal amount of adhesive on the slide corners, which was allowed to harden to keep the coverslip in place. Slides were mounted for fluorescence microscopy using Prolong™ Diamond Antifade Mounting Media (Invitrogen; cat. P36961), sealed with a rectangular #1.5 coverslip, and cured overnight before imaging.

### Collection of Microscopy Data

The slides as prepared above were imaged on an Olympus BX51 microscope connected to a Hamatsu ORCA-ER detector and illuminated with a Prior Lumen 200 source with a Hg lamp (Prior; cat LM200B1-A). Excitation lines and band pass (BP) or long pass (LP) emission filter pairs are as follows (excitation/BP or LP)-350 nm/450 nm BP; 405 nm/532 nm LP; 405 nm/610 nm LP. These pairs were used to capture PL from DAPI stained bacteria, CdSe-MPA QDs, and neither DAPI or CdSe-MPA QDs, respectively. The emission was collected through an infinity-corrected 60× PlanApo 1.4NA oil objective with a 0.17 mm working distance (Olympus). The emission is then passed through an OptiGrid structured illumination element to form a “grid” confocal image on the detector. For each sample, 12 images were taken at z-stack intervals of 0.5 μm for a total depth of 6 μm and compressed into the extended focus view presented in Figure S13. The exposure time for each channel was optimized and kept the same between each sample.

Time course lactate consumption was measured using a Colorimetric Lactate Assay Kit (Sigma Aldrich). Samples were prepared in a single well on a 96-well clear bottom plate using 2 mL of Lactate Enzyme Mix, 2 μL of Colorimetric Lactate Probe and 46 mL of Lactate Assay Buffer. The remaining 50 mL contained a lactic acid standard or minimal medium sample diluted with Lactate Assay Buffer. The samples were incubated for 30 min at room temperature. The samples were quantified using a Tecan Infinite 1000 multi-well plate reader using the absorbance at 570 nm. Samples (50 mL) were extracted from experimental vials at specific timepoints using a long sterile needle and aliquoted for quantification. Various dilutions of the minimal medium samples were used to ensure reproducibility of the absorbance readings. The external calibration was run with each assay to ensure accurate quantification.

### Collection of Colony Forming Unit Data.

Colony forming unit (CFU) data on the QD-bacteria aggregate was obtained by transferring the aggregate with a minimal amount of MM solution from the photochemical vial to a centrifuge tube and centrifuging at 1300 rpm for 2 min. The supernatant was removed, and the cell pellet was resuspended in 500 mL of 1× sterile PBS (pH 7.4). A series of 2-fold dilutions was performed to yield six total solutions. 100 mL of the solution was plated on an LB agar plate and incubated at 35° C. for 24 hrs. Plates with between 30 and 300 colonies were counted to yield CFU/mL quantities.

### Electron Microscopy Experiments.

For electron microscopy experiments, after a week-long hydrogen production experiment, aggregates comprised of MR-1 WT and CdSe-MPA were prepared for transmission electron microscopy imaging via fixation with a 2.5% glutaraldehyde/0.1 M sodium cacodylate solution. Images were taken on a Hitachi 7650 Transmission Electron Microscope with EDS (IF Instruments).

### Quantum Yield Measurements

The quantum yield for hydrogen generation was calculated according to the equation below:

$${Φ}_{{H}_{2}} = 2 \frac{k}{{q}_{p}}$$

Quantum yield (PH2) is equal to the amount of H₂ produced relative to photons absorbed. Two photons are required to produce one mole of H₂. k is the rate of H₂ production, defined as:

$$k = \frac{{H}_{2} ( mol )}{t ( s )}$$

qₚ is the photon flux, which measures of photons per second, and is defined as:

$${q}_{p} = \frac{n ( mol )}{t ( s )} = \frac{{P}_{abs} \times λ}{c \times h \times A}$$

The photon flux can be calculated using the power absorbed at a particular wavelength used during the experiment: P_{abs}=P₀−P_{QD}

In this case, P_{abs}(W), power absorbed, was calculated by subtracting the power when quantum dots were present, P_{QD}, from the initial power of the LED, P0, measured by a Newport power meter (Model 1918-C). For CdSe-MPA, the average P_{abs} is equal to 0.008 W at a wavelength (λ) of 525 nm. qp is calculated using the rest of the constants, where c is the speed of light (m/s), h is Planck's constant (J s), and A is Avogadro's constant (mol-1).

### Sample Calculation

H₂ Generated in 168 Hours: 20.8 μmol=2.1×10⁵ mol Time 168 hours=10,080 min=604,800 seconds

$$k = \frac{{H}_{2} ( mol )}{{t}_{t}} = \frac{( 2.1 \times {10}^{- 5} mol}{604800 s} ) = 3.5 \times {10}^{- 11} mol / s$$

$${q}_{p} = \frac{[ ( 0.008 \frac{J}{s} ) \times ( 525 \times {10}^{- 9} m ) ]}{[ ( 3 \times {10}^{8} \frac{m}{s} ) \times ( 6.626 \times {10}^{- 34} Js ) \times ( 6.022 \times {10}^{23} {mol}^{- 1} ) ]} = 3.5 \times {10}^{- 8} mol / s$$

$$\begin{matrix}{Φ}_{{H}_{2}} = \frac{2 \times ( 3.5 \times {10}^{- 10} mol / s )}{( 3.5 \times {10}^{- 8} \frac{mol}{s} )} \\ {Φ}_{{H}_{2}} = 0.2 \%\end{matrix}$$

### Results of QY Calculations

TABLE 5
Hydrogen production and quantum yield from light-driven
catalysis. A tabulation of average hydrogen evolved and
the corresponding TONs and QYs are shown in Table 5.

| | [CdSe-MPA] | Total H2 | | |
|---|---|---|---|---|
| Bacteria | (μM) | (μmol) | TONNC | QY (%) |
| WT MR-1 | 0 | 6.2 ± 3.2 | N/A | N/A |
| WT MR-1 | 1 | 18.2 ± 5.2 | 3,640 ± 1,040 | 0.17 ± 0.05 |
| ΔhyaA | 0 | 0 | N/A | N/A |
| ΔhyaA | 1 | 20.8 ± 5.8 | 4,160 ± 1,160 | 0.20 ± 0.05 |
| — | 1 | 0.8 ± 0.6 | 160 ± 120 | 0.08 ± 0.06 |

The nanoparticle may be any nanoparticle that is a semiconductor. Without intending to be bound by any particular theory, it is considered that, upon illumination by electromagnetic radiation, H₂ evolves from the nanoparticles, which are replenished by the bacteria through EET.

“Nanoparticle” (NP) as used herein includes nanocrystals, quantum dots (QDs), magic size clusters (MSCs), quantum rods, dot-in-rod nanocrystals, quantum wires, dendritic inorganic nanostructures, tetrapods, cubes, core-shell and alloy structures of any of the preceding, and the like. Nanoparticles also include any 3-D geometry whereby one or more dimensions are in nanoscale size (<100 nm). The nanocrystals include quantum dots such as, for example, cores, core-shells, alloyed cores, alloyed core-shells, and the like. Type I and Type II nanocrystals (e.g., Type II core-shell nanocrystals) can be used.

The nanoparticles can have a narrow size distribution. In an embodiment, a plurality of nanoparticles having a narrow size distribution (e.g., the nanoparticles are substantially monodisperse) is used. In an embodiment, the nanoparticles are substantially monodisperse. The term “substantially monodisperse” when describing nanoparticles denotes a population of nanoparticles of which a major portion, typically at least about 60%, in other embodiments from 75% to 90%, fall within a specified particle size range. A population of substantially monodisperse nanoparticles deviates 15% rms (root-mean-square) or less in diameter and typically less than 5% rms. In addition, upon exposure to a primary light source, a substantially monodisperse population of nanoparticles is capable of emitting energy in narrow spectral linewidths, as narrow as 12 nm to 60 nm full width of emissions at half maximum peak height (FWHM), and with a symmetric, nearly Gaussian line shape. The formulator will recognize, the linewidths are dependent on, among other things, the size heterogeneity (i.e., monodispersity) of the nanoparticles in each preparation.

The absorption profile and, thus, the reduction potential of the nanoparticles are determined, at least in part, by the size of the nanoparticles. A selected size (or size distribution) of nanoparticles can be used. In one embodiment, smaller nanoparticles are used. For example, in the case of CdSe nanoparticles, nanoparticles having a size of 1.5 nm to 30 nm, including all nm values and ranges there between, are used. In one example, CdSe QDs were synthesized with a diameter of 2.6 nm as defined by the wavelength of the peak in their lowest energy excitonic absorption feature (525 nm, FIG. 4). Quantum dots with varied sizes (between 2-10 nm) can be used. Changing the quantum dot size changes the wavelength of light absorbed and the energies of the conduction and valence bands. A higher conduction band may yield more active catalysis, and a lower valence band may enhance EET from bacteria.

The surface of the nanoparticles can be controlled. A desired surface composition (e.g., surface concentration of anions or cations) can be obtained using known methods. For example, known post-particle-formation reactions can be used. As another example, a desired surface composition can be obtained without using post particle formation reactions.

In an embodiment, the nanoparticles are capped (i.e., surface functionalized) with a ligand. The ligand can make the nanoparticles soluble in a convenient solvent (e.g., water). Such ligands are known in the art. Examples of suitable ligands include dihydrolipoic acid (DHLA), 3-mercapmercaptopropionic acid (e.g., MPA), and/or cysteine. Such capping ligands can be used with CdSe nanoparticles or other nanoparticles. In an embodiment, depending on the composition and/or structure of the nanoparticles, ligands are used that have one or more sulfur atoms. In another embodiment, ligands are used that have at least 2 sulfur atoms. Other ligands that can be used, particularly with CDSe QDs, are acids, amines and phosphines.

The nanoparticles can be doped with isovalent ions and/or aliovalent metal ions. The isovalent ions may be magnetic. Suitable metal ions include Mn²⁺, Cu²⁺, Co²⁺, Group I cations, Group III cations, Au⁺, Ag⁺, Al³⁺, and the like. Charged nanoparticles can be obtained using known nanoparticle formation reactions.

The nanoparticles can be obtained commercially or produced using methods known in the art. The nanoparticles can be soluble (or form a colloidal suspension) in an aqueous medium (e.g., water). For example, CdSe nanoparticles at a concentration of 0.5 M to 10 μM are used. Some portion of the nanoparticles can precipitate from the mixture and the mixture continues to produce hydrogen.

Examples of suitable nanoparticles include CdSe, CdS, CdTe, PbS, PbTe, PbSe, GaAs, InP, InAs, Si, and/or Ge nanoparticles. Suitable nanoparticles also include core-shell nanoparticles, including CdSe/CdS, CdSe/CdTe, CdTe/CdSe, and/or PbSe/PbS core-shell nanoparticles. Nanorods such as CdSe, CdS, CdTe, PbS, PbTe, PbSe, GaAs, InP, InAs, Si, and/or Ge nanorods may be used. Also suitable are dot-in rods, including CdSe/CdS, CdSe/CdTe, CdTe/CdS, and CdTe/CdSe dot-in rods. Zn-based II-VI core QDs are also suitable. Other examples include nanoplatelets such core-crown and core-shell nanoplatelets comprising CdSe, CdS, CdTe, PbS, PbTe, PbSe, GaAs, InP, InAs, Si, Ge, SnS, SnSe, SnTe, Ag2S, Ag2Se and/or Ag2Te. Suitable nanoparticles also include SnS, SnSe, SnTe, Ag2S, Ag2Se or Ag2Te. Combinations of any of the foregoing may also be suitable.

The nanoparticles may also be ternary or quaternary compounds. Cu-based ternary or quaternary quantum dots (QDs), or semiconductor nanocrystals (NCs) have unique optical properties regarding their emission mechanism, high photoluminescent quantum yields (PLQYs), size-dependent bandgap, composition-dependent bandgap, broad emission range, large Stokes' shift, and long photoluminescent (PL) lifetimes. Various types of Cu-based ternary or quaternary QDs (including anisotropic NCs, polytypic NCs, and spherical, nanorod and tetrapod core/shell heterostructures) may be used. For example, ternary Cu—In—Se (CISe) and Cu—In—S(CIS) compounds may be used, such as CuInS₂ and CuInSe₂, or Cu—Sn—Se (CTSe) and Cu—Sn—S(CTS) compounds may be used.

Additionally, metal halide perovskite NCs have attractive optical and electronic properties along with low cost and solution processability. Halide perovskites have a general formula of ABX₃, where A and B are monovalent and divalent cations, respectively, and X is a monovalent halide (Cl, Br, I) anion. Lead halide perovskites, classified into either organic-inorganic (hybrid) or all-inorganic, depending on whether the A cation is an organic molecule. Most commonly used is methylammonium (MA, CH₃NH₃₊) for a hybrid compound, or an inorganic cation (commonly Cs+), may be used for the inorganic compound. Examples are MAPbX₃ nanoparticles and caesium lead halide perovskite NCs (CsPbX₃) where X═Cl, Br or I.

In an embodiment, the nanoparticles are capped with a plurality of organic molecules (or mixture of organic molecules) and the catalyst has ligands formed from the same organic molecules. DHLA is an example of such an organic molecule.

Mixtures of nanoparticles can be used. The mixture can include, but is not limited to, nanoparticles having different sizes (e.g., average diameters) and/or different compositions. For example, in one embodiment, mixtures of two or more nanoparticles having different absorbance profiles are used. In another embodiment, mixtures of at least two nanoparticles having different absorbance profiles are used.

In an embodiment, the mixture of nanoparticles has a different absorption profile such that a greater portion of the solar spectrum is absorbed than is by one of the types of nanoparticles alone. For example, CdSe QDs can be used with QDs that absorb in the near infrared (e.g., CdTe, PbS, or PbSe) providing a nanoparticle mixture that absorbs in a greater portion of the solar spectrum than CdSe QDs alone.

The mixture of nanoparticles can be present in a variety of configurations. In an embodiment, two or more types (i.e., at least two types) of nanoparticles are mixed together in the aqueous medium. In another embodiment, multiple aqueous media (each having a different type of nanoparticle) are used. The multiple aqueous media are exposed to electromagnetic radiation (e.g., solar flux) in a desired sequence. For example, wider band gap particles absorb first letting the unabsorbed energy (e.g., infrared (IR) wavelengths) through to be absorbed by subsequent a narrower band gap particles (e.g., in a separate container).

A wide range of nanoparticle concentrations can be used. In one embodiment, to increase the production of hydrogen, the concentration(s) of the nanoparticle(s) is selected.

The source of electrons reduces the photoexcited nanoparticle. In one embodiment, the source of electrons is inexpensive. Internal sacrificial electron donors can be used. Such sacrificial electron donors can be present in the mixture. A fixed amount of a source of electrons can be used. For example, in one embodiment, a fixed amount of a source of electrons is an amount equal to the concentration of the nanoparticles up to the saturated amount the solution can hold. In another embodiment, a fixed amount of a source of electrons means an amount not continually added. Additional source(s) of electrons can be added to the mixture to provide a continuous hydrogen production. Hydrogen production can be continued as long as sufficient source of electrons is added or is available to the mixture. For example, the sacrificial electron donor (e.g., ascorbic acid) is present at concentrations of 0.1 M to 1 M, including all values to 0.1 M and ranges there between.

In an embodiment, the source of electrons is a biological system. Any biological system that can generate electrons can be used. In an embodiment, the biological system comprises prokaryotes that exhibit extracellular electron transfer (EET) such as, for example, electrogenic bacteria. The bacteria can be naturally-occurring bacteria or modified bacteria (e.g., genetically modified bacteria).

In an embodiment, the organisms consume a carbon source that the organisms then convert to electrons that are transferred extracellularly. The “carbon source” may include adenosine, L-arabinose, D-arabitol, D-galacturonic acid, inosine, D-psicose, uridine, 2′-deoxyadenosine, dihydroxyacetone, L-alanine, α-keto-valeric acid, L-lactic acid, D-lactic acid methyl ester, methylpyruvate, propionic acid, pyruvic acid, L-serine, acetic acid, acetoacetic acid, butyric acid, 2-amonoethanol, caproic acid, L-leucine, L-threonine, Tween 20, Tween 40, Tween 80, D-cellobiose, chondroitine sulfate C, α-cyclodextrin, β-cyclodextrin, γ-cyclodextrin, dulcitol, D-fructose, N-acetyl-D-glucosamine, N-acetyl-D-galactosamine, D-glucosamine, α-D-glucose, glycogen, laminarin, maltose, maltotriose, D-mannose, pectin, sucrose, Gly-Glu, Gly-Pro, Gly-Asp, Ala-Gly, gelatin, L-asparagine, bromosuccinic acid, citric acid, fumaric acid, L-glutamic acid, L-glutamine, L-isoleucine, α-ketobutyric acid, L-malic acid, D,L-malic acid, mono-methylsuccinate, phenylethylamine, putrescine, and/or succinic acid. These carbon sources are described in Rodrigues, J. L., Serres, M. H. & Tiedje, J. M. Large-scale comparative phenotypic and genomic analyses reveal ecological preferences of Shewanella species and identify metabolic pathways conserved at the genus level. Applied and Environmental Microbiology 77, 5352-5360 (2011). Preferably, the carbon source may be adenosine, 2′-deoxyadenosine, L-lactic acid, methylpyruvate, pyruvic acid, Tween 20, Tween 40, Tween 80, N-acetyl-D-glucosamine, Gly-Glu, Gly-Pro, Gly-Asp and/or gelatin. The L-lactic acid may be in the form of lactate. The carbon source may be found in, for example, bacterial growth media such as Luria-Bertani medium, organic matter, organic waste, or waste water from a natural or industrial source.

The biological system can be an internal source of electrons or an external source of electrons. For example, in an embodiment in which bacteria are the internal source of electrons, the bacteria make electrons and “spit” them out to the bacterial surface. These electrons then “bump” into nanoparticles to reduce them. In an embodiment in which bacteria are the external source of electrons, the bacteria reduce molecules in solution (e.g., flavins) that then reduce the nanoparticles.

Examples of suitable electrogenic bacteria include members of the Shewanella genus (e.g., Shewanella oneidensis and Shewanella putrefaciens), Aeromonas hydrophila, and genetically modified Escherichia coli (e.g., E. coli-MtrC), Shewanella oneidensis (e.g., Shewanella oneidensis MR-1) and Bacillus subtilis (e.g., Bacillus subtilis-Omc expressed and—T. potens EET-mediating proteins expressed). Other examples of suitable electrogenic bacteria include Gram-positive bacteria such as Gram-positive dissimilatory metal-reducing bacteria. Examples of such bacteria include Thermincola potens. Other examples are bacteria that excrete flavins, for example, Bacillus subtilis, and Bacillus megatarium. Another embodiment may use a Geobacter species. Mixtures of bacteria can be used. Shewanella oneidensis MR-1 in which genes for both hydrogenase enzymes are knocked out (ΔhydA/ΔhyaB) can be used, with the advantage that without their own functional hydrogenases, these strains will not consume hydrogen produced by the system.

Shewanella oneidensis in which genes expressing proteins that perform extracellular electron transfer are overexpressed will be used, with the advantage that these are expected to yield a greater flux of electrons to the quantum dots. The genes to be overexpressed include CymA, MtrA, MtrB, OmcA, MtrC.

There are two routes for electrons to get from bacteria to the NPs. One route is “direct” EET from the bacteria to the NP. The second route is the excretion of electron shuttles such as flavins that are then capable of reducing the oxidized NP. In both cases, the original source of electrons is whatever medium serves as nutrients for the bacteria.

In an embodiment, the source of electronic is electrogenic bacteria in the aqueous medium. In this embodiment, the electrogenic bacteria can be physically isolated from the QDs in the aqueous medium, but electrically connected to the QDs by a porous membrane (e.g., semi-permeable cellulose membranes, porous nanocrystalline silicon membranes, and porous carbon nanotube membranes) and a small molecule mediator (e.g., methyl viologen) added to facilitate electron transfer.

In another embodiment, the electron source is electrogenic bacteria disposed (e.g., as a biofilm comprising the bacteria) on an electrode. The electrode can be external or internal to the aqueous medium. The electrogenic bacteria may also adhere to an electrode or nanoparticles but not form a film.

The aqueous medium can have a variety of compositions. In an embodiment, the aqueous medium is water or wastewater. In an embodiment, the aqueous medium comprises water and/or wastewater. In another embodiment, the aqueous medium further comprises an organic solvent (or mixture of organic solvents). The organic solvent can be a protic solvent, an aprotic solvent, or a combination thereof. Examples of suitable organic solvents include alcohols such as ethanol, methanol, and ethylene glycol. For example, the aqueous medium is a 1:1 EtOH:water mixture by volume.

The pH of the medium can be from 2.0 to 11.0, including all pH values to 0.1 pH unit and ranges therebetween (i.e., the tolerance is to 0.1 pH unit). In one embodiment, to increase the production of hydrogen, the pH of the medium is selected. H₂ evolution may occur between pH 2.2 and 4.5. It may also occur at approximately pH 7 (method described in FIGS. 3A and 3B). “Approximately” means within +/−0.5 pH.

Any wavelength (or wavelength range) of electromagnetic radiation that forms a photoexcited nanoparticle can be used and can be easily determined by one skilled in the art. Depending on the nanoparticle, wavelengths in the ultraviolet, visible, and near infrared can be used. Wavelengths in the solar spectrum can be used. Wavelengths greater than the energy of the lowest unoccupied molecular orbital (LUMO) of the nanoparticle(s) can be used. Wavelengths greater than the bandgap energy of the nanoparticle(s) can be used.

The electromagnetic radiation can be provided in a variety of ways. Any radiation source providing the desired electromagnetic energy wavelength(s) can be used. For example, the electromagnetic radiation can be provided by a lamp (e.g., xenon lamp), arc lamp, black body radiation source, light emitting diode (LED), laser, or sunlight. The electromagnetic radiation can be provided in a continuous manner or intermittently as desired to control hydrogen production. Any range of wavelengths can be used as long as the nanoparticle absorbs the radiation. Preferably, the radiation comprises the visible and near-infrared (VNIR) portion of the electromagnetic spectrum, with wavelengths between approximately 400 and 1100 nanometers (nm). Most preferably, the radiation has a wavelength of 530 nm.

The nanoparticles, source of electrons, and aqueous medium mixture can be present in a vessel. The vessel allows exposure of the mixture to the desired electromagnetic radiation. The size of the vessel can be scaled to the desired rate of hydrogen production. Examples of suitable vessels include flasks, vials, and reactors. In the case where two or more (i.e., at least two) different nanoparticles are used, the vessel can be configured such that the different nanoparticles are physically separated from each other.

The mixture can be present in an inert atmosphere. For example, the mixture can be present in a nitrogen or argon atmosphere. The atmosphere can be a mixture of inert gases.

The methods can be carried out at a wide range of temperatures. The mixture can be at ambient temperature or elevated temperature. The method can be performed at ambient temperature or elevated temperature. By ambient temperature, it is meant a temperature of 15° C. (59° F.) to 25° C. (77° F.), though differences in climate may acclimate people to higher or lower temperatures. The mixture can be present at an elevated temperature of, for example, 25° C. to 40° C. The mixture can be present at approximately 25° C. The method can be performed at approximately 25° C. The generation of hydrogen can occur at approximately 25° C., at ambient temperature or elevated temperature.

In an embodiment, the composition and/or method produces hydrogen continuously for at least 164 hours without external voltage. Replenishing the carbon source allows catalysis to continue for a second week. In various embodiments, the method produces hydrogen for at least 100, 200, 300, or 400 hours without external voltage.

Nano-bio H₂-evolving assembly designs. Three distinct assemblies can be used.

The first approach is direct combination of QDs and bacteria in solution (FIG. 11A). In this system, the sacrificial electron donor is replaced by electrogenic bacteria, and the system is run in an appropriate bacterial medium. As bacteria respire, they engage in EET. The electrons are then available for H₂ production which occurs through respiration of the QDs through EET. This assembly can be constructed with and without soluble redox mediators. Because electrogenic bacteria typically donate electrons directly to solid metals, and have been shown to donate electrons to nanoparticles, direct electron transfer from bacteria to QDs is expected. The electrons produced by electrogenic bacteria are expected to have sufficient reducing power to serve as donors to oxidized QDs. Indeed, typical reduction potentials of OMCs (~−0.3 V) are significantly lower than that of ascorbate (~0.06 V). A number of bacterial strains may be used in this assembly including S. oneidensis MR-1, which is frequently used in biofuel cells and has well-understood EET function, and engineered E. coli-MTR, expressing EET-mediating proteins of S. oneidensis MR-1.

In addition, Gram-positive dissimilatory metal-reducing bacteria can be used. Such bacteria are attractive microbes because of their resistance to QD-mediated toxicity. An example is Thermincola potens, which undergoes EET via OMCs. It is expected that B. subtilis can be engineered to express the T. potens EET-mediating proteins. An advantage of using B. subtilis is that it already shows wide use for protein expression not only in research laboratories but also for a range of industrial applications, and is capable of excreting recombinant proteins.

The second approach that can be used is separating the electrogenic bacteria and QDs by a porous membrane (FIG. 11B) that allows the passage of small molecules and ions but not bacteria and QDs. In this scheme, QDs and bacteria do not come into direct contact with each other. Charge is transferred using a small-molecule mediator added to the system; some bacteria such as Shewanella or Bacillus also excrete mediators such as flavins. The advantage of this approach is that any damage to bacteria that may result from direct contact with QDs will be mitigated, although the cost may be lower efficiency owing to the use of electron mediators. Nevertheless, small-molecule electron shuttles are routinely used in MFCs and MECs with success, although a direct electron-transfer approach can be used when feasible.

The third approach is to construct a photo-bioelectrochemical cell (FIG. 11C) in which electrogenic bacteria deliver electrons to the anode, and QDs receive electrons from the cathode to catalyze H₂ evolution. The electrons are generated in situ from the bacteria, as opposed to coming external to the system such as from an applied potential. Irradiation of the QDs, putting them into the excited state, provides sufficient energy needed to drive the reaction, as has already been established. The QDs and bacteria can be separated by a cation-exchange membrane; Nafion™ is commonly used in BECs. This setup parallels that of MECs, except that no external voltage need be applied, because the needed energy is supplied photochemically. An advantage of this system is that the separation of the QDs and bacteria mitigates concerns about QD toxicity. Furthermore, biofilm-forming bacteria can be grown directly on the anode for direct electron transfer, which increases efficiency relative to use of mediators.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

While embodiments of the present disclosure have been particularly shown and described with reference to certain examples and features, it will be understood by one skilled in the art that various changes in detail may be effected therein without departing from the spirit and scope of the present disclosure as defined by claims that can be supported by the written description and drawings. Further, where exemplary embodiments are described with reference to a certain number of elements it will be understood that the exemplary embodiments can be practiced utilizing either less than or more than the certain number of elements.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

1. A composition for producing hydrogen, consisting essentially of:
(a) a nanoparticle or plurality of nanoparticles, selected from the group consisting of: (1) CdSe, CdS, CdTe, PbS, PbTe, PbSe, GaAs, InP, InAs, Si, and Ge nanoparticles; (2) core-shell nanoparticles, including CdSe/CdS, CdSe/CdTe, CdTe/CdSe, and PbSe/PbS core-shell nanoparticles; (3) CdSe, CdS, CdTe, PbS, PbTe, PbSe, GaAs, InP, InAs, Si, and Ge nanorods; (4) dot-in rods, including CdSe/CdS, CdSe/CdTe, CdTe/CdS, and CdTe/CdSe dot-in rods; (5) Zn-based II-VI core QDs, (6) nanoplatelets including core-crown and core-shell nanoplatelets comprising CdSe, CdS, CdTe, PbS, PbTe, PbSe, GaAs, InP, InAs, Si, Ge, SnS, SnSe, SnTe, Ag2S, Ag2Se and/or Ag2Te; (7) SnS, SnSe, SnTe, Ag2S, Ag2Se and Ag2Te; and combinations thereof;
(b) an external source of electrons, wherein the external source of electrons is an electrogenic bacterium or a plurality of electrogenic bacteria; and
(c) a bacterial growth medium;
wherein the nanoparticle or the plurality of nanoparticles and the bacterial growth medium are combined in a mixture;
upon exposure to electromagnetic radiation with a wavelength in the absorption profile of the nanoparticle or the plurality of nanoparticles, the nanoparticle or the plurality of nanoparticles is capable of generating an electron that can reduce a proton in the bacterial growth medium; and the source of electrons is capable of reducing the nanoparticle or the plurality of nanoparticles.

2. (canceled)

3. The composition of claim 1, wherein the nanoparticle or the plurality of nanoparticles comprise cadmium chalcogenide (CdSe or CdS) or water-soluble cadmium chalcogenide (CdSe or CdS) quantum dots.

4. (canceled)

5. The composition of claim 1, wherein the electrogenic bacterium or plurality of electrogenic bacteria comprise Shewanella oneidensis, a Geobacter species or any bacterium capable of extracellular electron transfer.

6. The composition of claim 5, wherein the electrogenic bacterium or plurality of electrogenic bacteria comprise Shewanella oneidensis and have at least one mutation selected from the group consisting of (1) deletion of the genes HydA and/or HyaB, encoding hydrogenases; (2) deletion of any of the hydrogenase-related genes, or any combination of HydA, HydB, HydE, HydF, and/or HydG; (3) deletion or overexpression of any of the genes associated with extracellular electron transfer, including OmcA, MtrA, MtrB, MtrC, and/or CymA; (4) deletion or overexpression of any of the genes associated with extracellular electron transfer mediated by flavins and/or flavin biosynthesis, including RibF, RibDBAE, and/or OprF; (5) deletion or overexpression of any of the genes associated with biofilm formation including RibF and/or YdeH; and (6) display of surface peptides containing amino acids that may bind to nanoparticles, including histidine and cysteine.

7. (canceled)

8. (canceled)

9. The composition of claim 1, wherein the nanoparticle or the plurality of nanoparticles comprise water-soluble cadmium chalcogenide (CdSe or CdS) quantum dots, and wherein the electrogenic bacterium or plurality of electrogenic bacteria comprise Shewanella oneidensis, a Geobacter species or a genetically-modified bacterium.

10. (canceled)

11. (canceled)

12. (canceled)

13. (canceled)

14. The composition of claim 9, wherein the electrogenic bacterium or plurality of electrogenic bacteria comprise Shewanella oneidensis and have at least one mutation selected from the group consisting of (1) deletion of the genes HydA and/or HyaB, encoding hydrogenases; (2) deletion of any of the hydrogenase-related genes, or any combination of HydA, HydB, HydE, HydF, and/or HydG; (3) deletion or overexpression of any of the genes associated with extracellular electron transfer, including OmcA, MtrA, MtrB, MtrC, and/or CymA; (4) deletion or overexpression of any of the genes associated with extracellular electron transfer mediated by flavins and/or flavin biosynthesis, including RibF, RibDBAE, and/or OprF; (5) deletion or overexpression of any of the genes associated with biofilm formation including RibF and/or YdeH; and (6) display of surface peptides containing amino acids that may bind to nanoparticles, including histidine and cysteine.

15. (canceled)

16. (canceled)

17. (canceled)

18. (canceled)

19. (canceled)

20. A method for producing hydrogen consisting essentially of:
(a) providing a source of electrons wherein the source of electrons is an electrogenic bacterium or a plurality of electrogenic bacteria;
(b) contacting a nanoparticle or a plurality of nanoparticles in a bacterial growth medium to form a mixture in the presence of the source of electrons, wherein the nanoparticle or plurality of nanoparticles are selected from the group consisting of: (1) CdSe, CdS, CdTe, PbS, PbTe, PbSe, GaAs, InP, InAs, Si, and Ge nanoparticles; (2) core-shell nanoparticles, including CdSe/CdS, CdSe/CdTe, CdTe/CdSe, and PbSe/PbS core-shell nanoparticles; (3) CdSe, CdS, CdTe, PbS, PbTe, PbSe, GaAs, InP, InAs, Si, and Ge nanorods; (4) dot-in rods, including CdSe/CdS, CdSe/CdTe, CdTe/CdS, and CdTe/CdSe dot-in rods; (5) Zn-based II-VI core QDs, (6) nanoplatelets including core-crown and core-shell nanoplatelets comprising CdSe, CdS, CdTe, PbS, PbTe, PbSe, GaAs, InP, InAs, Si, Ge, SnS, SnSe, SnTe, Ag2S, Ag2Se and/or Ag2Te; (7) SnS, SnSe, SnTe, Ag2S, Ag2Se and Ag2Te; and combinations thereof; and
(c) exposing the mixture from (b) to electromagnetic radiation having at least a wavelength in the absorption profile of the nanoparticle or the plurality of nanoparticles, wherein:
upon exposure to the electromagnetic radiation, the nanoparticle or the plurality of nanoparticles is capable of generating an electron that can reduce a proton in the bacterial growth medium, and
the source of electrons is capable of reducing the nanoparticle or the plurality of nanoparticles, such that hydrogen is produced.

21. (canceled)

22. The method of claim 20, wherein the nanoparticle or the plurality of nanoparticles comprise cadmium chalcogenide (CdSe or CdS) or water-soluble cadmium chalcogenide (CdSe or CdS) quantum dots.

23. (canceled)

24. The method of claim 20, wherein the electrogenic bacterium or plurality of electrogenic bacteria comprise Shewanella oneidensis, a Geobacter species or any bacterium capable of extracellular electron transfer.

25. The method of claim 24, wherein the electrogenic bacterium or plurality of electrogenic bacteria comprise Shewanella oneidensis and have at least one mutation selected from the group consisting of (1) deletion of the genes HydA and/or HyaB, encoding hydrogenases; (2) deletion of any of the hydrogenase-related genes, or any combination of HydA, HydB, HydE, HydF, and/or HydG; (3) deletion or overexpression of any of the genes associated with extracellular electron transfer, including OmcA, MtrA, MtrB, MtrC, and/or CymA; (4) deletion or overexpression of any of the genes associated with extracellular electron transfer mediated by flavins and/or flavin biosynthesis, including RibF, RibDBAE, and/or OprF; (5) deletion or overexpression of any of the genes associated with biofilm formation including RibF and/or YdeH; and (6) display of surface peptides containing amino acids that may bind to nanoparticles, including histidine and cysteine.

26. (canceled)

27. (canceled)

28. (canceled)

29. (canceled)

30. (canceled)

31. The method of claim 20, wherein the nanoparticle or the plurality of nanoparticles comprise water-soluble cadmium chalcogenide (CdSe or CdS) quantum dots, and the electrogenic bacterium or plurality of electrogenic bacteria comprise Shewanella oneidensis, a Geobacter species or any bacterium capable of extracellular electron transfer.

32. The method of claim 31, wherein the electrogenic bacterium or plurality of electrogenic bacteria comprise Shewanella oneidensis and have at least one mutation selected from the group consisting of (1) deletion of the genes HydA and/or HyaB, encoding hydrogenases; (2) deletion of any of the hydrogenase-related genes, or any combination of HydA, HydB, HydE, HydF, and/or HydG; (3) deletion or overexpression of any of the genes associated with extracellular electron transfer, including OmcA, MtrA, MtrB, MtrC, and/or CymA; (4) deletion or overexpression of any of the genes associated with extracellular electron transfer mediated by flavins and/or flavin biosynthesis, including RibF, RibDBAE, and/or OprF; (5) deletion or overexpression of any of the genes associated with biofilm formation including RibF and/or YdeH; and (6) display of surface peptides containing amino acids that may bind to nanoparticles, including histidine and cysteine.

33. (canceled)

34. (canceled)

35. (canceled)

36. (canceled)

37. (canceled)

38. A composition for producing hydrogen, comprising:
(a) a nanoparticle or plurality of nanoparticles, wherein the nanoparticle or the plurality of nanoparticles are selected from the group consisting of: (1) CdSe, CdS, CdTe, PbS, PbTe, PbSe, GaAs, InP, InAs, Si, and Ge nanoparticles; (2) core-shell nanoparticles, including CdSe/CdS, CdSe/CdTe, CdTe/CdSe, and PbSe/PbS core-shell nanoparticles; (3) CdSe, CdS, CdTe, PbS, PbTe, PbSe, GaAs, InP, InAs, Si, and Ge nanorods; (4) dot-in rods, including CdSe/CdS, CdSe/CdTe, CdTe/CdS, and CdTe/CdSe dot-in rods; (5) Zn-based II-VI core QDs, (6) nanoplatelets including core-crown and core-shell nanoplatelets comprising CdSe, CdS, CdTe, PbS, PbTe, PbSe, GaAs, InP, InAs, Si, Ge, SnS, SnSe, SnTe, Ag2S, Ag2Se and/or Ag2Te; (7) SnS, SnSe, SnTe, Ag2S, Ag2Se and Ag2Te; and
combinations thereof;
(b) an external source of electrons, wherein the external source of electrons is an electrogenic bacterium or a plurality of electrogenic bacteria;
(c) a surface; and
(d) a bacterial growth medium;
wherein the nanoparticle or the plurality of nanoparticles adhere to the surface and the surface is in contact with the bacterial growth medium;
upon exposure to electromagnetic radiation with a wavelength in the absorption profile of the nanoparticle or the plurality of nanoparticles, the nanoparticle or the plurality of nanoparticles is capable of generating an electron that can reduce a proton in the bacterial growth medium; and the source of electrons is capable of reducing the nanoparticle or the plurality of nanoparticles.

39. The composition of claim 38, wherein the surface is an electrode or is comprised of quantum dots and wherein at least one electrogenic bacterium adheres to the surface.

40. The composition of claim 39, wherein electrogenic bacteria form a biofilm.

41. (canceled)

42. The composition of claim 38, wherein the nanoparticle or the plurality of nanoparticles comprise cadmium chalcogenide (CdSe or CdS) or water-soluble cadmium chalcogenide (CdSe or CdS) quantum dots.

43. (canceled)

44. The composition of claim 38, wherein the electrogenic bacterium or plurality of electrogenic bacteria comprise Shewanella oneidensis, a Geobacter species or any bacterium capable of extracellular electron transfer.

45. The composition of claim 38, wherein the nanoparticle or the plurality of nanoparticles comprise water-soluble cadmium chalcogenide (CdSe or CdS) quantum dots, and wherein the electrogenic bacterium or plurality of electrogenic bacteria comprise Shewanella oneidensis, a Geobacter species or any bacterium capable of extracellular electron transfer.

46. The composition of claim 44, wherein the wherein the electrogenic bacterium or plurality of electrogenic bacteria comprise Shewanella oneidensis and have at least one mutation selected from the group consisting of (1) deletion of the genes HydA and/or HyaB, encoding hydrogenases; (2) deletion of any of the hydrogenase-related genes, or any combination of HydA, HydB, HydE, HydF, and/or HydG; (3) deletion or overexpression of any of the genes associated with extracellular electron transfer, including OmcA, MtrA, MtrB, MtrC, and/or CymA; (4) deletion or overexpression of any of the genes associated with extracellular electron transfer mediated by flavins and/or flavin biosynthesis, including RibF, RibDBAE, and/or OprF; (5) deletion or overexpression of any of the genes associated with biofilm formation including RibF and/or YdeH; and (6) display of surface peptides containing amino acids that may bind to nanoparticles, including histidine and cysteine.

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. The composition of claim 40, wherein the nanoparticle or the plurality of nanoparticles comprise cadmium chalcogenide (CdSe or CdS) or water-soluble cadmium chalcogenide (CdSe or CdS) quantum dots.

60. The composition of claim 40, wherein the electrogenic bacterium or plurality of electrogenic bacteria comprise Shewanella oneidensis, a Geobacter species or any bacterium capable of extracellular electron transfer

61. The composition of claim 40, wherein the nanoparticle or the plurality of nanoparticles comprise water-soluble cadmium chalcogenide (CdSe or CdS) quantum dots, and wherein the electrogenic bacterium or plurality of electrogenic bacteria comprise Shewanella oneidensis, a Geobacter species or any bacterium capable of extracellular electron transfer.
